(19) United States
(12) Patent Application Publication
(10) Pub. No.: US 2011/0250675 A1
(43) Pub. Date: Oct. 13, 2011

(54) Virus-Like Particle Mediated Cellular Delivery

(75) Inventor: Robert Bennett, Encinitas, CA (US)

(73) Assignee: LIFE TECHNOLOGIES CORPORAITON, Carlsbad, CA (US)

(21) Appl. No.: 12/934,628

(22) PCT Filed: Mar. 26, 2009
(86) PCT No.: PCT/US09/38431

§ 371 Date: May 25, 2011

Related U.S. Application Data
(60) Provisional application No. 61/039,692, filed on Mar. 26, 2008.

(51) Int. Cl. C12N 7/00; C12N 1/00; C12N 5/07; C12N 5/04

(52) U.S. Cl. 435/235.1; 435/410; 435/243; 435/375

(57) ABSTRACT

The invention provides compositions and methods for delivering compounds to cells. The invention is directed, in part, to virus-like particles which contain biological materials such as carbohydrates, proteins and nucleic acids. The invention is also directed, in part, to methods for delivering compounds to cells involving contacting cells with the compounds under conditions which allow for uptake of the compounds by cells and release of the compounds into the cells which take it up.

### FIELD OF THE INVENTION

The invention provides compositions and methods for delivering compounds to cells. The invention is directed, in part, to virus-like particles which contain biological materials such as carbohydrates, proteins and nucleic acids. The invention is also directed, in part, to methods for delivering compounds to cells involving contacting cells with the compounds under conditions which allow for uptake of the compounds by cells and release of the compounds into the cells which take it up.

### BACKGROUND OF THE INVENTION

The delivery of compounds to cells is often limited by the fact that trafficking of many compounds into living cells is restricted by cellular membrane systems. In many instance, specific transporters allow the selective entry of nutrients or regulatory molecules, while excluding most exogenous molecules such as nucleic acids and proteins. Various strategies can be used to improve transport of compounds into cells, including the use of lipid carriers, biodegradable polymers, and various conjugate systems.

Some methods for improving the transport of foreign nucleic acids, for example, into cells involve the use of viral vectors or cationic lipids and related cytofectins. Viral vectors can be used to transfer genes efficiently into some cell types, but attempts to use such vectors to introduce chemically synthesized molecules into cells have been less successful.

Another approach to delivering biologically active molecules involves the use of conjugates. Conjugates are often selected based on the ability of certain molecules to be selectively transported into specific cells, for example, via receptor-mediated endocytosis. By attaching a compound of interest to molecules that are actively transported across the cellular membranes, the effective transfer of that compound into cells or specific cellular organelles can be realized. Alternately, molecules that are able to penetrate cellular membranes without active transport mechanisms, for example, various lipophilic molecules, can be used to deliver compounds of interest. Examples of molecules that can be utilized as conjugates include but are not limited to peptides, hormones, fatty acids, vitamins, flavonoids, sugars, reporter molecules, reporter enzymes, chelators, porphyrins, intercalcators, and other molecules that are capable of penetrating cellular membranes, either by active transport or passive transport.

A number of peptide based cellular transporters have also been developed. These peptides are capable of crossing cellular membranes in vitro and in vivo with high efficiency. Examples of such fusogenic peptides include a 16-amino acid fragment of the homeodomain of antennapedia, a Drosophila transcription factor (Wang et al., Proc. Natl. Acad. Sci. USA, 92:3318-3322 (1995)); a 17-mer fragment representing the hydrophobic region of the signal sequence of Kaposi fibroblast growth factor with or without NLS domain (Antopolsky et al., Bioconj. Chem., 10:598-606 (1999)); a 17-mer signal peptide sequence of a caiman crocodylus immunoglobin light chain (Chaloin et al., Biochem. Biophys. Res. Comm., 243:601-608 (1997)); a 17-amino acid fusion sequence of HIV envelope glycoprotein gp4114, (Morris et al., Nucleic Acids Res., 25:2730-2736 (1997)); the HIV-1 Tat49-57 fragment (Schwarze et al., Science, 285:1569-1572 (1999)); and others.

Another approach to the intracellular delivery of biologically active molecules involves the use of cationic polymers. For example, Ryser et al., PCT Publication No. WO 79/00515 describes the use of high molecular weight lysine polymers for increasing the transport of various molecules across cellular membranes. Further, Rothbard et al., PCT Publication No. WO 98/52614, describes certain methods and compositions for transporting drugs and macromolecules across biological membranes in which the drug or macromolecule is covalently attached to a transport polymer consisting of from 6 to 25 subunits, at least 50% of which contain a guanidino or amidino side chain. Polyarginine peptides composed of all D-, all L- or mixtures of D- and L-arginine have been shown to work particularly well. Rothbard et al., U.S. Patent Publication No. 2003/0082356, describes certain poly-lysine and poly-arginine compounds for the delivery of drugs and other agents across epithelial tissues, including the skin, gastrointestinal tract, pulmonary epithelium and blood brain barrier.

Another approach to the intracellular delivery of biologically active molecules involves the use of liposomes or other particle forming compositions. Since the first description of liposomes in 1965, by Bangham (J. Mol. Biol. 13:238-252), there has been a sustained interest and effort in the area of developing lipid-based carrier systems for the delivery of pharmaceutically active compounds. Liposomes are attractive drug carriers since they protect biological molecules from degradation while improving their cellular uptake. One of the most commonly used classes of liposome formulations for delivering polyanions (e.g., DNA) is that which contains cationic lipids. Lipid aggregates can be formed with macromolecules using cationic lipids alone or including other lipids and amphiphiles such as phosphatidylethanolamine. Both the composition of the lipid formulation as well as its method of preparation are know to have effect on the structure and size of the resultant anionic macromolecule-cationic lipid aggregate. These factors can be modulated to optimize delivery of polyanions to specific cell types in vitro and in vivo. The use of cationic lipids for cellular delivery of biologically active molecules has several advantages. The encapsulation of anionic compounds using cationic lipids is essentially quantitative due to electrostatic interaction. In addition, it is believed that the cationic lipids interact with the negatively charged cell membranes initiating cellular membrane transport (Akhtar et al., Trends Cell Bio., 2:139 (1992); Xu et al., Biochemistry 35:5616 (1996)).

Recombinant viruses are currently used for a wide variety of applications. Viruses may be used for medical applications, for example, in gene therapy applications and/or as vaccines. Viruses may also be used in biotechnology applications, for example, as vectors to clone nucleic acids of interests and/or to produce proteins. Examples of recombinant viruses that have been used include, but are not limited to, herpes viruses (see, for example, Kelly et al., U.S. Pat. No. 5,672,344), pox viruses such as vaccinia virus (see, for example, Moss et al., 1997, in Current Protocols in Molecular Biology, Chapters 16.15-16.18, John Wiley & Sons), papilloma viruses (see, for example, Bruck et al., U.S. Pat. No. 6,342,224), retroviruses (see, for example, Chavez et al., U.S. Pat. No. 6,300,118), adenoviruses (see, for example, Crouzet et al., U.S. Pat. No. 6,261,807), adeno-associated viruses (AAV, see for example, Srivastava, U.S. Pat. No. 5,252,479), and coxsackie viruses (see, for example, U.S. Pat. No. 6,323,024).

When the viral nucleic acid is not infectious, as with, for example, certain pox viruses, construction of recombinant viruses may involve in vivo homologous recombination in a virus-infected cell between the viral genome and concomitantly transfected plasmid bearing a sequence of interest flanked by viral sequences. When the viral nucleic acid is infectious, as with, for example, certain adenoviruses, a modified viral nucleic acid may be prepared and transfected into a host cell.

Methods for constructing recombinant viruses are typically laborious and time consuming. There remains a need in the art for materials and methods for convenient and efficient construction of viral vectors designed to deliver molecules to cells. This need and others are met by the present invention. Thus, the present application provides, in part, compounds, compositions and methods for delivering compounds (e.g., RNA molecules) to cells.

### SUMMARY OF THE INVENTION

The invention provides, in part, compositions and methods for delivering compounds to cells. The invention is directed, in part, to virus-like particles (VLPs) which are associated with (e.g., contain) biological materials such as lipids, carbohydrates, proteins and nucleic acids. Other compounds which may be associated with VLPs include dyes (e.g., fluorescent dyes), labels (e.g., fluorescent or radioactive labels), and drugs (e.g., antibiotics or anti-viral agents). The invention is also directed, in part, to methods for delivering compounds to cells involving contacting the cells with compounds under conditions which allow for uptake of the compounds by these cells and/or intracellular release of the compounds. Thus, the invention is directed, in part, to compositions and methods for delivering one or more (e.g., one, two, three, four, five, etc.) compounds to cells. In many embodiments, the invention includes a VLP with which the compound(s) to be delivered are associated with (e.g., contained in).

As explained in more detail elsewhere herein, the invention also includes VLPs and components of VLPs which are designed for use in forming compositions discussed herein, as wells as use in methods discussed herein. As an example, the invention includes modified VLP components which are designed to bind to compounds and facilitate their association with VLPs which have these components.

In particular embodiments, the invention is directed to methods for introducing nucleic acid molecules (e.g., RNA or DNA) into cells (e.g., prokaryotic or eukaryotic cells). In some aspects such methods can comprise: (a) selecting a nucleic acid of interest which is heterologous to the cells; (b) transcribing the nucleic acid of interest to generate an RNA molecule; (c) forming virus-like particles under conditions which result in the RNA molecule being incorporated into the virus-like particles; and (d) contacting the cell with the virus-like particles formed in step (c). In particular instances, the nucleic acid molecule may not contain a packaging signal. The invention further comprises compositions made by such methods (e.g., cells which contain compounds).

In additional particular instances, nucleic acid molecules (e.g., heterologous nucleic acid molecules) associated with VLPs in various aspects of the invention may be of particular sizes. Examples of such sizes include, less than about 20, less than about 25, less than about 30, less than about 35, less than about 40, less than about 45, less than about 50, less than about 55, less than about 65, less than about 70, less than about 75, less than about 80, less than about 90, less than about 100, less than about 125, or less than about 150 nucleotides in length. Further, exemplary ranges of such sizes include from about 10 to about 300 nucleotides, from about 10 to about 25 nucleotides, from about 10 to about 30 nucleotides, from about 15 to about 25 nucleotides, from about 15 to about 30 nucleotides, from about 20 to about 25 nucleotides, from about 20 to about 30 nucleotides, from about 21 to about 27 nucleotides, from about 22 to about 26 nucleotides, from about 20 to about 300 nucleotides, from about 20 to about 200 nucleotides, from about 20 to about 150 nucleotides, from about 20 to about 100 nucleotides from about 25 to about 150 nucleotides, from about 25 to about 100 nucleotides, from about 25 to about 95 nucleotides, from about 25 to about 90 nucleotides, from about 25 to about 80 nucleotides, from about 25 to about 70 nucleotides, from about 25 to about 60 nucleotides, from about 25 to about 50 nucleotides, from about 25 to about 40 nucleotides, from about 30 to about 100 nucleotides, from about 35 to about 100 nucleotides, from about 40 to about 100 nucleotides, from about 50 to about 100 nucleotides, from about 60 to about 100 nucleotides, from about 70 to about 100 nucleotides, from about 80 to about 100 nucleotides, from about 80 to about 95 nucleotides, and from about 80 to about 90 nucleotides.

In particular aspects of the invention, VLPs employed may contain components from particular viruses. Such viruses include viruses which are specific for prokaryotic or eukaryotic host. Exemplary categories of viruses include phage, baculoviruses, adenoviruses, adeno-associated viruses, lentiviruses, pox viruses, and alphaviruses. It should be noted that viruses are obligate intracellular parasites which typically introduce their nucleic acid into cells. Along these lines, the invention is directed, in part, to methods and compositions employing one or more virus to transfer compounds (e.g., heterologous compounds) into cells. Thus, in particular aspects, the invention employs at least some natural property or properties of viruses for desired purposes. In particular embodiments of the invention, the VLPs are generated using components from retroviruses such as a Moloney Murine leukemia virus and/or a lentivirus.

Any number of compounds may be associated with VLPs in the practice of the invention. For example, these compounds may be nucleic acids such as DNA or RNA or mixtures of DNA and RNA. Nucleic acids used in the invention may be single-stranded, double-stranded or may even be in other forms such as triplexes. Further, when nucleic acids are in a form other than single-stranded (e.g., double-stranded), these nucleic acids may be composed of one nucleic acid stranded or more than one nucleic acid strand (e.g., two separate molecules of RNA of DNA). When nucleic acid molecules are composed of one strand with a double-stranded region, these molecules may form a hairpin. Hairpins will typically have a double-stranded region connected by a single-stranded region which forms a loop connecting nucleic acid regions with sequence complementarity. Often, the loop is processed in vivo to form two separate strands. In many instances, such double-stranded regions will have sequence complementarity such that they hybridize to each other under stringent hybridization conditions.

Sizes of regions of sequence complementarity and loops can vary greatly. In many instances, regions of sequence complementarity in nucleic acid molecules of the invention may be of varying size, including from about 10 to about 300 nucleotides, from about 15 to about 300 nucleotides, from about 18 to about 300 nucleotides, from about 20 to about 300 nucleotides, from about 21 to about 300 nucleotides, from about 22 to about 300 nucleotides, from about 25 to about 300 nucleotides, from about 50 to about 300 nucleotides, from about 60 to about 300 nucleotides, from about 70 to about 300 nucleotides, from about 80 to about 300 nucleotides, from about 90 to about 300 nucleotides, from about 100 to about 300 nucleotides, from about 110 to about 300 nucleotides, from about 10 to about 200 nucleotides, from about 10 to about 110 nucleotides, from about 10 to about 100 nucleotides, from about 10 to about 90 nucleotides, from about 10 to about 80 nucleotides, from about 10 to about 70 nucleotides, from about 10 to about 60 nucleotides, from about 10 to about 50 nucleotides, from about 10 to about 40 nucleotides, from about 10 to about 30 nucleotides, from about 18 to about 300 nucleotides, from about 18 to about 110 nucleotides, from about 18 to about 100 nucleotides, from about 18 to about 90 nucleotides, from about 18 to about 70 nucleotides, from about 18 to about 50 nucleotides, from about 18 to about 40 nucleotides, from about 18 to about 30 nucleotides, from about 20 to about 300 nucleotides, from about 20 to about 110 nucleotides, from about 20 to about 100 nucleotides, from about 20 to about 90 nucleotides, from about 20 to about 75 nucleotides, from about 20 to about 50 nucleotides, from about 20 to about 40 nucleotides, from about 20 to about 30 nucleotides, from about 20 to about 28 nucleotides, from about 20 to about 26 nucleotides, from about 22 to about 120 nucleotides, from about 22 to about 110 nucleotides, from about 22 to about 90 nucleotides, from about 22 to about 80 nucleotides, from about 22 to about 60 nucleotides, from about 22 to about 50 nucleotides, from about 22 to about 40 nucleotides, from about 22 to about 30 nucleotides, from about 22 to about 28 nucleotides, from about 23 to about 40 nucleotides, from about 23 to about 30 nucleotides, from about 23 to about 28 nucleotides, from about 50 to about 300 nucleotides, from about 50 to about 125 nucleotides, from about 50 to about 110 nucleotides, from about 50 to about 100 nucleotides, from about 50 to about 90 nucleotides, from about 50 to about 80 nucleotides, from about 50 to about 70 nucleotides, from about 60 to about 125 nucleotides, or from about 68 to about 120 nucleotides.

Loops, when present, in nucleic acid molecules of the invention may be of varying size, including from about 3 to about 50 nucleotides, from about 4 to about 50 nucleotides, from about 5 to about 50 nucleotides, from about 6 to about 50 nucleotides, from about 7 to about 50 nucleotides, from about 8 to about 50 nucleotides, from about 9 to about 50 nucleotides, from about 10 to about 50 nucleotides, from about 3 to about 10 nucleotides, from about 4 to about 10 nucleotides, from about 5 to about 10 nucleotides, from about 6 to about 10 nucleotides, from about 7 to about 10 nucleotides, from about 8 to about 10 nucleotides, from about 4 to about 12 nucleotides, from about 4 to about 14 nucleotides, from about 4 to about 15 nucleotides, from about 4 to about 18 nucleotides, from about 4 to about 20 nucleotides, from about 4 to about 25 nucleotides, from about 5 to about 15 nucleotides, from about 6 to about 14 nucleotides, from about 5 to about 12 nucleotides, or from about 6 to about 12 nucleotides.

The invention also includes methods for inhibiting gene expression, as well as compositions which may be used in such methods. In particular embodiment, the invention includes methods of inhibiting expression of a gene of interest, these methods may comprise, (a) selecting the gene of interest; (b) generating a nucleic acid molecule (e.g., an RNA molecule) with sequence complementarity to a transcript corresponding to the gene of interest; (c) forming virus-like particles under conditions which result in the nucleic acid molecule being incorporated into the virus-like particles; and (d) contacting the cell with the virus-like particles formed in step (c). In specific embodiments, the nucleic acid molecule may not contain a packaging signal. In additional specific embodiments, the nucleic acid molecule will be of a length described herein (e.g., less than 150 nucleotides in length).

The nature of the gene of interest will vary greatly with the particular application. For example, the gene of interest may encode either a functional RNA or a polypeptide (e.g., expressed from a mRNA). Examples of functional RNAs include transfer RNA and ribosomal RNA. Examples of polypeptides are numerous and include cytokines, transcription factors, receptors, etc.

When the compound for which cellular delivery is desired is an RNA, this RNA may be of any type. RNA molecules which may be delivered to cells by VLPs (or encoded by nucleic acid associated with VLPs include (a) microRNAs; (b) short hairpin RNAs; (c) short interfering RNAs, and (d) messenger RNAs (mRNAs).

The invention also includes methods for preparing virus-like particles which contain compounds (e.g., nucleic acid molecules such as DNA and/or RNA molecules). In particular embodiments, such methods include those which can comprise: (a) selecting one or more compounds for which cell delivery is desired; (b) generating the compounds, and (c) forming virus-like particles under conditions which result in the compound being incorporated into the virus-like particles. In some instances, methods of the invention will further comprise contacting a cell with a virus-like particles which is associated with (e.g., contains) one or more compound. In particular instances where the compound is a nucleic acid, the nucleic acid may share sequence complementarity, identity, or similarity to a transcript corresponding to a gene of interest. In many such instances, knock-down of expression of the gene of interest will result from contacting of a cell with the VLPs.

In specific aspects, the invention includes methods for producing VLPs which contain one or more nucleic acid molecules (e.g., RNA or DNA). In specific embodiments, such methods may comprise (a) selecting one or more nucleic acid of interest; (b) transcribing the nucleic acid of interest to generate one or more RNA molecules; and (c) forming VLPs under conditions which result in the RNA molecules being incorporated into the VLPs. In related embodiments, VLPs may be associated with various types of nucleic acids (e.g., heterologous nucleic acids) such as DNA, RNA, both RNA and DNA, or RNA/DNA hybrids.

In additional specific embodiments, the invention includes methods for producing VLPs which contain one or more RNA molecules, these methods comprising (a) selecting a nucleic acid of interest; (b) synthesizing the one or more RNA molecules with sequence identity to one or more nucleic acid of interest; and (c) forming virus-like particles under conditions which result in the RNA molecules being incorporated into the virus-like particles.

The invention also includes methods of knocking-down the expression of a gene in target cells. In specific embodiments, methods of the invention include those comprising: (a) selecting one or more gene which is expressed in a target cell for which knock-down is desired; (b) generating one or more nucleic acid molecules designed to and/or capable of knocking-down gene expression when introduced into the target cell; (c) forming VLPs under conditions which result in the nucleic acid molecule of step (b) being incorporated into the VLPs; and (d) contacting the target cell with the VLPs formed in step (c).

The invention further includes compositions which comprising a population of VLPs, wherein one or more members of the population of virus-like particles are associated with (e.g., contain) at least one heterologous compound (e.g., at least one heterologous nucleic acid molecule which does not contain a packaging signal). The number of varying features of VLPs of this aspect of the invention is considerable. For examples, the population of VLPs may vary in the number of compound molecules per VLP or the number of VLPs in the population which are associated with compounds (e.g., one or more identical or different compound molecules).

Using nucleic acid molecules as an exemplary category of heterologous compounds, the invention includes populations of VLPs in which greater than 1% (e.g., from about 1% to about 12%, from about 1% to about 25%, from about 5% to about 95%, from about 10% to about 95%, from about 20% to about 95%, from about 30% to about 95%, from about 45% to about 95%, from about 60% to about 95%, from about 75% to about 95%, from about 5% to about 85%, from about 5% to about 75%, from about 5% to about 65%, from about 5% to about 55%, from about 5% to about 45%, from about 5% to about 35%, from about 5% to about 25%, from about 5% to about 15%, from about 20% to about 80%, from about 20% to about 70%, from about 20% to about 60%, from about 20% to about 50%, or from about 30% to about 60%), 5%, 10%, 20%, 30%, 40%, 50%, 60%, 70%, or 80% of the individual members of the population each are associated with (e.g., contain) at least one (e.g., one, two, three, four, five, etc.) heterologous nucleic acid molecule. Of course, the numbers referred to above, as well as elsewhere herein, apply to VLPs (e.g., populations of VLPs) which are associated with compounds other than nucleic acid molecules.

The invention also includes populations of VLPs in which a certain number or percentage of members of the population of VLPs each are associated with two or more of the same or different compounds (e.g., heterologous compounds). There are at least two variations of this aspect of the invention. First, there is the number or percentage of VLPs which contain two or more compounds. Second, there is the number of compounds associated with individual VLPs in the population. For example, if 75% of the VLPs in a population are associated with no compound molecules and 25% of the VLPs are each associated with one compound molecule, then it can be said that there are, on average, 0.25 compound molecules per VLP. As another example, if 25% of the VLPs in a population are associated with no compound molecules, 50% of the VLPs are each associated with one compound molecule, and 25% of the VLPs are each associated with two compound molecules, then it can be said that there are, on average, 0.75 compound molecules per VLP.

In many instances, the average number of compounds per VLP will be from about 0.05 to about 5.0, from about 0.1 to about 5.0, from about 0.2 to about 5.0, from about 0.5 to about 5.0, from about 0.5 to about 5.0, from about 0.7 to about 5.0, from about 0.9 to about 5.0, from about 1.0 to about 5.0, from about 1.5 to about 5.0, from about 2.0 to about 5.0, from about 2.5 to about 5.0, from about 3.0 to about 5.0, from about 3.5 to about 5.0, from about 0.05 to about 4.0, from about 0.05 to about 3.5, from about 0.05 to about 3.0, from about 0.05 to about 2.5, from about 0.05 to about 2.0, from about 0.05 to about 1.5, from about 0.05 to about 1.0, from about 0.05 to about 0.7, from about 0.2 to about 4.0, from about 0.2 to about 2.0, from about 0.2 to about 1.0, from about 0.5 to about 4.0, from about 0.5 to about 3.0, from about 0.5 to about 2.0, from about 0.5 to about 1.0, from about 1.0 to about 4.0, from about 1.0 to about 3.0, from about 1.0 to about 2.0, from about 1.5 to about 4.0, or from about 1.5 to about 2.5.

In some instances, at least 1%, 5%, 10%, 20%, 30%, 40%, 50%, 60%, 70%, or 80% of the members of the population of virus-like particles each contain two or more (e.g., from about three to about twenty, from about three to about fifteen, from about three to about ten, from about three to about eight, from about three to about six, from about two to about twenty, from about two to about ten, from about two to about five, from about five to about twenty, from about five to about fifteen, from about seven to about twenty, from about seven to about fifteen, from about seven to about twelve, from about nine to about twenty, etc.) different heterologous nucleic acid molecules or other compound molecules.

The invention further includes VLPs in one or more component has been altered from their wild-type form, as well as the individual components themselves and methods for using VLPs which contain such components for delivering compounds to cells. Thus, in specific aspects, the invention includes compositions which can comprise (a) a virus-like particle containing at least one non-wild-type component which has been selected to introduce, remove, enhance or diminish one or more property; and (b) a heterologous compound. In many instances, the property which has introduced, removed, enhanced or diminished will result in a change in binding affinity for the heterologous compound. In many instances, the non-wild-type component will be a polypeptide which contains one or more (e.g., one, two, three, four, five, six, etc.) amino acid alterations as compared to the wild-type component. As one skilled in the art would recognize, such alterations may be substitutions, deletions, and/or insertions. Thus, two separate deletions of fifteen and ten contiguous amino acids is two alterations.

When the invention employs nucleic acid molecules, these nucleic acid molecules may contain one or more chemical modifications. As explained elsewhere herein in more detail, these modifications may be anywhere in the nucleic acid molecules, such as between one or more sugar residue of the backbone. Specific chemical modifications which may be employed in the practice of the invention include 2′-O-propyl modification, 2′-O-methyl modifications, 2′-O-ethyl modifications, and 2′-fluoro modifications. In particular embodiments, nucleic acid molecules may contain at least one (e.g., one, two, three, four, five, six, seven, eight, nine, ten, twelve, fourteen, sixteen, eighteen, etc.) 2′-fluoro modification and at least one e.g., one, two, three, four, five, six, seven, eight, nine, ten, twelve, fourteen, sixteen, eighteen, etc.) 2′-O-methyl modification. In additional particular embodiments, nucleic acid molecules may contain at least one (e.g., one, two, three, four, five, six, seven, eight, nine, ten, twelve, fourteen, sixteen, eighteen, etc.) 2′-O-methyl modifications. When more than one chemical modification is present in a double-stranded nucleic acid molecules, these modifications may be present on one strand or both strands.

In many instances, when chemical modifications are present on nucleic acid molecules used in the invention, these nucleic acid molecules will be chemically synthesized.

### BRIEF DESCRIPTION OF THE DRAWINGS

FIG. 1 shows a general overview of particular aspects of the invention. In embodiments of the invention described in this figure, nucleic acid molecules which encode components of virus-like particles (VLPs) are introduced into a cell, also introduced into the cell are either (A) nucleic acid molecules which encode one or more compounds, represented by the short bars, and other short nucleic acid molecules, represented by dotted lines, or (B) the one or more compounds themselves. When VLPs are generated in and released from the cell, at least some of the VLPs contain one or more compound molecule. The dashed circle around the VLPs indicates than an envelope is present. In this instance, eight compound molecules are shown in five VLPs. Thus, the average number of compound molecules is 1.6 per VLP.

FIG. 2. Lentiviral or viral-like-particle delivery of shRNA targeting lacZ genes knockdown the β-galactosidase activity in HT1080 cells. HT1080 cells transiently expressing lacZ gene were infected with lentiviruses or virus-like particles and analyzed β-galactosidase activity 24 hours later. The β-galactosidase activity was normalized to total protein of cells.

FIG. 3. Lentiviral or viral-like-particle delivery of shRNA targeting lacZ genes knockdown the β-galactosidase activity in GripTite 293 cells. GripTite 293 cells transiently expressing lacZ gene were infected with lentiviruses or virus-like particles and analyzed β-galactosidase activity 24 hr later. The β-galactosidase activity was normalized to luciferase activity in the cells.

FIG. 4. QPCR analysis of lacZ expression in 293 Cells. Flp-In 293 cells stably expressing lacZ were transduced with various amounts of lentiviral particles containing shRNA (pLP/shRNA) or with lentivirus expressing shRNAs in transduced cells pLenti6.2 lacZ). Cells harvested at 24 or 48 hours post transduction were analyzed for lacZ mRNA levels by qPCR.

FIG. 5. Cytotoxicity assay of viral particle preparations. Flp-In 293 cells stably expressing lacZ were transduced with various amounts of lentiviral particles containing shRNA (pLP/shRNA), lentivirus without shRNAs (empty particle) or with lentivirus that express shRNAs in transduced cells (pLenti6.2 lacZ). Media from cells at 24 hours was analyzed using the Vybrant Cytoxicity Assay Kit-G6PD release assay (Invitrogen, Carlsbad, Calif.).

### DETAILED DESCRIPTION OF THE INVENTION

### Definitions

Unless defined otherwise, all technical and scientific terms used herein have the same meanings as commonly understood by one of ordinary skill in the art to which this invention belongs.

As used herein, the term “virus-like particle” or “VLP” refers to a vehicle for delivering one or more compounds into cells. VLPs will contain at least one viral protein. Typically, with VLPs, viral protein will surround the compounds. However, in particular instances, compounds can be associated with a VLP by means other than inclusion in the VLP. For example, compounds may be attached (e.g., covalently or non-covalently attached) to a viral protein or integrated into the envelope, when present.

Examples of VLPs include viral particle products produced by using VIRAPOWER™ adenoviral and lentiviral vector kits (see, e.g., Invitrogen Corporation, cat. nos. K4930-00, K4940-00, K4950-00, K4955-00, K4960-00, K4965-00, K4967-00, and K4985-00).

Viruses which may be used to prepare VLPs include, for examples, phage, (e.g., T even phages (e.g., T4 phage, etc.), T odd phage (e.g., T7 phage, etc.), bacteriophage phi29, lambda phage, etc.), baculoviruses, adenoviruses, adeno-associated viruses, lentiviruses (e.g., Moloney Murine leukemia virus, HIV1, HTLV-III, etc.), pox viruses, and alphaviruses (e.g., Semliki Forest Virus, SindBis Virus, etc.). Additional examples of viruses which may be used to prepare VLPs, as well as methods for preparing VLPs are described elsewhere herein.

Viruses which may be used to prepare VLPs include those with double-stranded or single-stranded genomes, RNA or DNA genomes, and enveloped or non-enveloped viruses. As explained in more detail elsewhere herein, when VLPs contain an envelope, the envelop may be used to deliver lipids, polypeptides, carbohydrates, and other compounds to cells.

As used herein, the term “compound” refers to a material which can be delivered to a cell by a VLP. Examples of compounds include biological monomers and polymers such as polypeptides, nucleic acids (e.g., DNA, RNA, etc.), carbohydrates, and lipids. In many instances, nucleic acids will be contained within VLPs. Compounds other than nucleic acid may often be associated with VLPs by being contain within the VLP or by association with a VLP polypeptide or other constituent (e.g., a lipid of an envelope).

As used herein, the term “selecting”, when used in respect to a compound for delivery by a VLP or gene for knock-down, refers to the identification of the compound. As an example, when one seeks to knock-down the expression of a gene using an RNAi molecule or a microRNA molecule, the gene of interest is chosen for knock-down. Thus, the selection here represents a conscious decision to identify and then knock down expression of the particular gene. Typically, in such an instance, one would identify, the gene for knock-down and then would select a nucleic acid molecule intended to knock-down that gene. Thus, in this instance, the molecule which mediates knock-down, not the gene for which knock-down is desired, is selected. When a compound is selected for delivery to a cell by a VLP, the compound is identified as one for which cellular delivery is desired.

As used herein, the term “heterologous”, when used in regards to a cell or a VLP, refers to something which is not normally associated with the cell or VLP in nature. For example, when a microRNA is generated in a cell from an engineered nucleic acid, the microRNA is heterologous to the cell and, if incorporated into a VLP, is also heterologous to the VLP.

As used herein, the term “double-stranded”, when used in reference to a nucleic acid molecule, refers to the molecule having a region where nucleotides are hybridized to each other. Thus, a double-stranded nucleic acid molecule may be composed on a single molecule with at least two regions which will hybridize to each other either under physiological conditions or stringent conditions or two separate molecules each of which with at least one region which will hybridize to each other either under physiological conditions or stringent conditions. Thus, a “hairpin turn” nucleic acid molecule is considered to be double-stranded. Typically, double-stranded regions of a double-stranded nucleic acid molecule will be at least 10, 15, 20, 25, 30, 40, 50, 75, 80, 90, 100, 120, or 140 nucleotides in length.

As used herein, the term “single-stranded”, when used in reference to a nucleic acid molecule, refers to a nucleic acid molecule which is not hybridized to another nucleic acid molecule and has no regions which will hybridize intramolecularly either under physiological conditions or stringent condition. As one skilled in the art would understand, nucleic acid molecules can have double-stranded and single-stranded regions.

As used herein, the term “adenovirus” refers to a DNA virus of the Adenoviridae family. As one skilled in the art would recognize, a considerable number of human adenovirus (mastadenovirus H) immunotypes exist, including Type 1 through 42 (including 7a).

As used herein, the term “retrovirus” refers to a virus which alternates between RNA and DNA forms. Examples of such viruses include lentiviruses. Specific examples of retroviruses include Moloney Murine leukemia virus (MoMuLV or MMLV), Harvey Murine sarcoma virus (HaMuSV or HSV), Murine mammary tumor virus (MuMTV or MMTV), gibbon ape leukemia virus (GaLV or GALV), human immunodeficiency viruses (HIV) (e.g., HIV-1, HIV-2, etc.), and Rous sarcoma virus (RSV).

As used herein, the term “baculovirus” refers to members of a family of large rod-shaped viruses which is typically divided into two sub-groups: (1) nucleopolyhedroviruses (NPV) and (2) granuloviruses (GV). While GVs generally contain only one nucleocapsid per envelope, NPVs generally contain either single (SNPV) or multiple (MNPV) nucleocapsids per envelope. Generally, the enveloped virions are further occluded in granulin matrix in GVs and polyhedrin for NPVs. Baculoviruses have very species-specific tropisms among the invertebrates with over 600 host species having been described. Immature (larval) forms of moth species are the most common hosts, but these viruses have also been found infecting sawflies, mosquitoes, and shrimp.

As used herein, the term “Togavirus” refers to a family of viruses, including the following: Alphaviruses (e.g., Sindbis virus, Eastern equine encephalitis virus, Western equine encephalitis virus, Venezuelan equine encephalitis virus, Ross River virus, O'nyong'nyong virus, etc.), Rubiviruses (e.g., Rubella virus)

Togaviruses typically have a genome which is composed of linear, single-stranded, positive sense RNA. The 5′-terminus often carries a methylated nucleotide cap and the 3′-terminus has a polyadenylated tail, therefore resembling cellular mRNA. These viruses are often enveloped and form spherical particles (65-70 nm diameter). The capsid is typically icosahedral and constructed of 240 monomers, having a triangulation number of 4. Normally, after virus attachment and entry into the cell, gene expression and replication takes place within the cytoplasm.

Togaviruses non-structural proteins are typically encoded at the 5′ end, formed during the first of two characteristic rounds of translation. These proteins may be originally translated as a polyprotein, which consequently undergo self cleavage, forming a number (e.g., four) non-structural proteins responsible for gene expression and replication. Typically, a sub-genomic fragment is formed which encodes the structural proteins and a negative sense fragment. Viral particle assembly typically takes place at the cell surface, where the virus buds from the cell, acquiring the envelope, when present.

As used herein, the term “adeno-associated virus” (AAV) is the smallest of known human viruses. These viruses can incorporate themselves into the host cell's genome and thus presents a very attractive subject for creating vectors for gene therapy.

The AAV genome is composed of single-stranded deoxyribonucleic acid (ssDNA), either positive- or negative-sensed, which is typically about 4.7 kilobase long. The genome normally comprises inverted terminal repeats (ITRs) at both ends of the DNA strand, and two open reading frames (ORFs): rep and cap. The former is generally composed of four overlapping genes encoding Rep proteins required for the AAV life cycle, and the latter generally contains overlapping nucleotide sequences of capsid proteins: VP1, VP2 and VP3, which interact together to form a capsid of an icosahedral symmetry. At least eleven serotypes of AAV are known.

As used herein, the term “bacteriophage” refers to viruses which use bacteria as hosts. Examples of bacteriophage include λ phage, T4 phage, T7 phage, R17 phage, M13 phage, MS2 phage, G4 phage, P1 phage, P2 phage, N4 phage, Φ6 phage, and Φ29 phage.

As used herein, the term “gene” refers to nucleic acid which contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. Of course, the definition of a “gene” does not include nucleic acid which encodes the transcriptional and translational machinery necessary to produce a functional product. Also, excluded are items such as transcription factors which induce transcription of, for example, a particular mRNA When the gene encodes an untranslated RNA, it includes the promoter and the nucleic acid that encodes the untranslated RNA.

As used herein, the phrase “structural gene” refers to refers to nucleic acid which is transcribed into messenger RNA, which is then translated into a sequence of amino acids characteristic of a specific polypeptide.

As used herein, the term “host” refers to any prokaryotic or eukaryotic cell or organism (e.g., a bacterial cell, a mammalian cell, an insect cell, a yeast cell, a plant cell, an avian cell, an animal cell, a protozoan cell, etc.) which is a recipient of a VLP and/or a nucleic acid molecule. In many instances, a nucleic acid molecule will be delivered to the host. These nucleic acid molecules may contain, but a not limited to, a nucleic acid segment or gene of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

As used herein, the phrase “transcriptional regulatory sequence” refers to a functional stretch of nucleotides contained on a nucleic acid molecule, in any configuration or geometry, that act to regulate the transcription of (1) one or more structural genes (e.g., two, three, four, five, seven, ten, etc.) into messenger RNA or (2) one or more genes into untranslated RNA. Examples of transcriptional regulatory sequences include, but are not limited to, promoters (e.g., RNA polymerase I promoters, RNA polymerase II promoters such as the CMV promoter, and RNA polymerase III promoters such as the H1 promoter and the U6 promoter), enhancers, repressors, operators (e.g., the tet operator), and the like. Transcriptional regulatory sequences used in the practice of the invention may share sequence homolog or identity with transcriptional regulatory sequence obtained from any source (e.g., the promoters of the human H1 gene).

As used herein, a “promoter” is an example of a transcriptional regulatory sequence, and is specifically a nucleic acid generally described as the 5′-region of a gene located proximal to the start codon or nucleic acid that encodes untranslated RNA. The transcription of an adjacent nucleic acid segment is initiated at or near the promoter. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

As used herein, the term “nucleic acids” (which is used herein interchangeably and equivalently with the term “nucleic acid molecules”) refers to nucleic acids (including DNA, RNA, and DNA-RNA hybrid molecules) that are isolated from a natural source; that are prepared in vitro, using techniques such as PCR amplification or chemical synthesis; that are prepared in vivo, e.g., via recombinant DNA technology; or that are prepared or obtained by any appropriate method. Nucleic acids used in accordance with the invention may be of any shape (linear, circular, etc.) or topology (single-stranded, double-stranded, linear, circular, supercoiled, torsional, nicked, etc.). The term “nucleic acids” also includes without limitation nucleic acid derivatives such as peptide nucleic acids (PNAs) and polypeptide-nucleic acid conjugates; nucleic acids having at least one chemically modified sugar residue, backbone, internucleotide linkage, base, nucleotide, nucleoside, or nucleotide analog or derivative; as well as nucleic acids having chemically modified 5′ or 3′ ends; and nucleic acids having two or more of such modifications. Not all linkages in a nucleic acid need to be identical.

As used herein, the term “nucleotide” refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [α-S]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both RNA and DNA.

As used herein, the term “oligonucleotide” refers to a synthetic or natural molecule comprising a covalently linked sequence of nucleotides that are joined by a phosphodiester bond between the 3′ position of the pentose of one nucleotide and the 5′ position of the pentose of the adjacent nucleotide.

As used herein, the term “polypeptide” refers to a sequence of contiguous amino acids of any length. The terms “peptide,” “oligopeptide,” or “protein” may be used interchangeably herein with the term “polypeptide.”

As used herein, the terms “hybridization” and “hybridizing” refer to base pairing of two complementary single-stranded nucleic acid molecules (RNA and/or DNA) to give a double-stranded molecule. As used herein, two nucleic acid molecules may hybridize, although the base pairing is not completely complementary. Accordingly, mismatched bases do not prevent hybridization of two nucleic acid molecules provided that appropriate conditions, well known in the art, are used. In some aspects, hybridization is said to be under “stringent conditions.” By “stringent conditions,” as the phrase is used herein, is meant overnight incubation at 42° C. in a solution comprising: 50% formamide, 5×SSC (750 mM NaCl, 75mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

As used herein, “transduce” and “transduction” refer to a process of introducing a virus into a cell type that does not support replication of the virus and does not result in the production of infectious viral progeny. In contrast, “infect” or “infection” are used to indicate introduction of a virus into a cell type that supports replication and results in the production of infectious viral progeny.

Other terms used in the fields of recombinant nucleic acid technology and molecular and cell biology as used herein will be generally understood by one of ordinary skill in the applicable arts.

### Overview of Aspects of the Invention

The invention is directed, in part, to methods and compositions for delivering compounds to cells. The invention will typically have or employ one or more of the following features: (1) identification of a gene for which knock-down or overexpression is desired, (2) selection of a compound for which introduction into a cell is desired (e.g., to facilitating gene knock-down or gene product overexpression), (3) production of a compound (e.g., a selected compound) for cellular delivery, (4) contacting of a cell with the compound, and/or (5) collection of data related to one or more effects the compound has on the cell. Thus, the invention will typically be directed to methods and compositions either containing or relating to one or more of the features referred to above. Of course, additional features may also be employed.

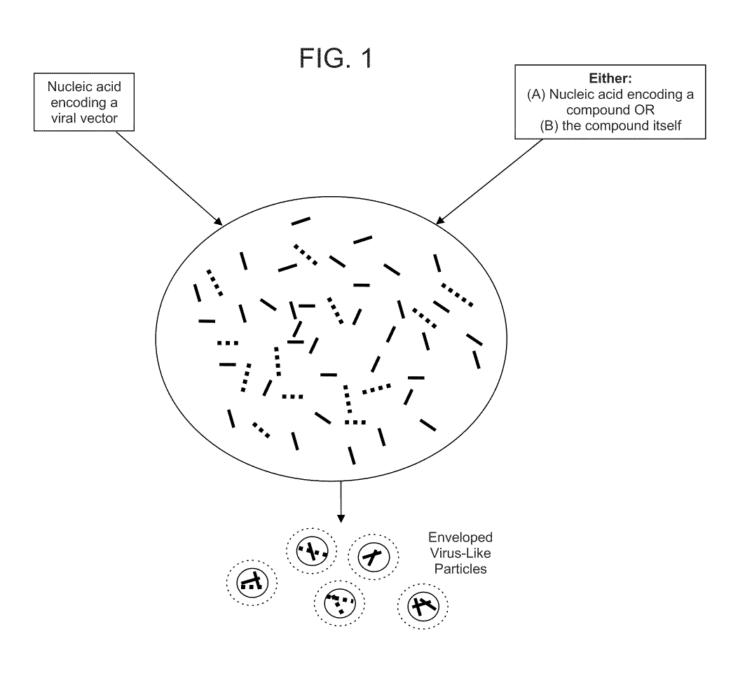

One general aspect of the invention is described in FIG. 1. In this aspect, VLPs are sought to be formed which contain a particular nucleic acid molecule (shown as short black bars). Other nucleic acid is shown as dotted lines. Thus, VLP encoding nucleic acid which encodes components of a VLP are introduced into a cell. Also introduced into the cell is either nucleic acid molecules which encode the particular nucleic acid molecule for introduction into the VLPs or the particular nucleic acid molecules for incorporation themselves. Upon formation of the VLPs, some of the particular nucleic acid molecules are contained in VLPs which are released from the cell. Of course, FIG. 1 refers to particular nucleic acid molecules only as an example. Thus, other compounds may be substituted for particular nucleic acid molecules.

In many instances, the particular nucleic acid molecules may or may not contain a packaging sequence. When a packaging sequence is not present, the number of VLPs which contain particular nucleic acid molecules and the number of particular nucleic acid molecules in the VLPs (e.g., the average number of particular nucleic acid molecules in the VLPs) will vary with a number of factors. Examples of such factors include the number of particular nucleic acid molecules within the cell, the number of particular nucleic acid molecules per unit area within the cell, the size of the particular nucleic acid molecules, the number of VLPs formed, and the intracellular nuclease activity/stability of the particular nucleic acid molecule. Other features, which will generally be more relevant when the compound is not a nucleic acid molecule, include the net and/or local charge of the compound, the polarity of the compound (e.g., hydrophobicity or hydrophilicity), the net and/or local structure of the compound (e.g., linear, globular, etc.), the affinity of the compound to associate with other atoms or molecules (e.g., the ability of the compound to form dimers, trimers and/or aggregates), and the affinity of the compound to associate with one or more VLP components (e.g., a capsid protein or a protein localized in the VLP's envelope, when present). Thus, a number of factors are believed to effect whether and how much of a compound is associated with VLPs. Examples of such factors include the size of the compound (e.g., the molecular weight), the shape of the compound (e.g., linear, globular, etc.), the local concentration of the compound where VLPs are formed, and attractive forces between one or more VLP components and the compound (e.g., affinity of a VLP protein for the compound). Thus, to some extent, the association of compounds with VLPs is believed to be concentration dependent. For sake of illustration, if VLPs are formed in two different cells which contain the same compound but there is more of the compound in one cell at the site where VLPs are forming, in many instance, the cell with the higher concentration of the compound will be expected to yield (1) VLPs which contain more compound molecules and/or (2) a population of VLPs where a higher percentage of the individual members contain the compound.

Along the lines of the above, methods of the invention may be employed, for example to produce VLPs which, on average contain from about 1 to about 1,000 compound molecules (e.g., from about 1 to about 400, from about 2 to about 400, from about 3 to about 400, from about 1 to about 10, from about 1 to about 30, from about 4 to about 400, from about 4 to about 20, from about 6 to about 40, from about 5 to about 50, from about 5 to about 100, from about 15 to about 200, from about 15 to about 400, from about 15 to about 1,000, from about 50 to about 1,000, from about 100 to about 1,000, from about 200 to about 1,000, from about 400 to about 1,000, from about 500 to about 1,000, etc.

Also, in many instances, VLPs will contain compound molecules of different types. For example, VLPs may contain particular nucleic acid molecules and other nucleic acid molecules which are normally found within cells (see, e.g., FIG. 1). Thus, VLPs generated by methods of the invention, and hence VLPs of the invention, may contain a ratio of compound molecules to other molecules of between from about 1:0.1 to 1:1,000 (e.g., from about 1:0.1 to 1:500, from about 1:0.1 to 1:400, from about 1:0.1 to 1:300, from about 1:0.1 to 1:200, from about 1:0.1 to 1:100, from about 1:0.1 to 1:50, from about 1:0.1 to 1:10, from about 1:0.1 to 1:5, from about 1:1 to 1:1,000, from about 1:1 to 1:500, from about 1:1 to 1:400, from about 1:1 to 1:200, from about 1:1 to 1:100, from about 1:1 to 1:50, from about 1:1 to 1:25, from about 1:1 to 1:10, from about 1:10 to 1:1,000, from about 1:10 to 1:500, from about 1:10 to 1:250, from about 1:10 to 1:100, from about 1:20 to 1:1,000, from about 1:20 to 1:500, from about 1:20 to 1:150, etc.). Typically, these other molecules will not include items such as salts and water but will include proteins, carbohydrates, and nucleic acids. In many instances, the above ratios will be determined with respect to other molecules of a similar type to the compound molecules. Thus, if the VLPs contain a compound molecule which is a protein, the other molecules would also be proteins. Further, if the VLPs contain a compound molecule which is a nucleic acid, the other molecules would also be nucleic acids.

### Compounds

Any number of compounds may be used in the practice of the invention. Examples of such compounds include non-polymeric and polymeric molecules. Biological monomers and polymers which may be used in the practice of the invention include polypeptides, nucleic acids (e.g., DNA, RNA, etc.), dyes, drugs, carbohydrates, and lipids. Exemplary descriptions of compounds which may be used in the practice of the invention are set out below.

A. Nucleic Acids

With respect to nucleic acids, compounds may vary by any number of features including type (e.g., DNA, RNA, etc.), size (e.g., length, molecular weight, total number of nucleotides, etc.), nucleotide sequence, base pair composition (e.g., having a particular C:G to A:T/U ratio, etc.) strandedness (e.g., double-stranded, single-stranded, partially double-stranded, partially, single-stranded, fully or partially triplexed, etc.), internucleoside phosphate backbone structure (e.g., having one or more phosphtioates, etc.), base modifications (e.g., having one or more 2′-O-propyl, 2′-O-methyl, 2′-O-ethyl, and/or 2′-fluoro modifications, etc.).

(1) Short RNA Molecules and Other Nucleic Acids

Nucleic acids can be synthesized either in vivo or in vitro, prepared from natural biological sources (e.g., cells, organelles, viruses and the like), or collected as an environmental or other sample. Examples of nucleic acids include without limitation oligonucleotides (including but not limited to antisense oligonucleotides), ribozymes, aptamers, polynucleotides, artificial chromosomes, cloning vectors and constructs, expression vectors and constructs, gene therapy vectors and constructs, PNA (peptide nucleic acid) DNA and RNA. VLPs may contain any of these nucleic acids.

RNA includes without limitation rRNA, mRNA, and Short RNA. As used herein, the term “Short RNA” encompasses RNA molecules described in the literature as “tiny RNA” (Storz, Science 296:1260-3, 2002; Illangasekare et al., RNA 5:1482-1489, 1999); prokaryotic “small RNA” (sRNA) (Wassarman et al., Trends Microbiol. 7:37-45, 1999); eukaryotic “noncoding RNA (ncRNA)”; “micro-RNA (microRNA)”; “small non-mRNA (smRNA)”; “functional RNA (fRNA)”; “transfer RNA (tRNA)”; “catalytic RNA” [e.g., ribozymes, including self-acylating ribozymes (Illangaskare et al., RNA 5:1482-1489, 1999]; “small nucleolar RNAs (snoRNAs)”; “tmRNA” (a.k.a. “10S RNA”, Muto et al., Trends Biochem. Sci. 23:25-29, 1998; and Gillet et al., Mol. Microbiol. 42:879-885, 2001); RNAi molecules including without limitation “small interfering RNA (siRNA)”, “endoribonuclease-prepared siRNA (e-siRNA)”, “short hairpin RNA (shRNA)”, and “small temporally regulated RNA (stRNA)”; “diced siRNA (d-siRNA)”, and aptamers, oligonucleotides and other synthetic nucleic acids that comprise at least one uracil base.

(2) Oligonucleotides

As used in the present invention, an oligonucleotide is a synthetic or biologically produced molecule comprising a covalently linked sequence of nucleotides which may be joined by a phosphodiester bond between the 3′ position of the pentose of one nucleotide and the 5′ position of the pentose of the adjacent nucleotide. As used herein, the term “oligonucleotide” includes natural nucleic acid molecules (i.e., DNA and RNA) as well as non-natural or derivative molecules such as peptide nucleic acids, phosphorothioate-containing nucleic acids, phosphonate-containing nucleic acids and the like. In addition, oligonucleotides of the present invention may contain modified or non-naturally occurring sugar residues (e.g., arabinose) and/or modified base residues. The term oligonucleotide encompasses derivative molecules such as nucleic acid molecules comprising various natural nucleotides, derivative nucleotides, modified nucleotides or combinations thereof. Oligonucleotides of the present invention may also comprise blocking groups which prevent the interaction of the molecule with particular proteins, enzymes or substrates.

Oligonucleotides include without limitation RNA, DNA and hybrid RNA-DNA molecules. Further, oligonucleotides may be of essentially any length referred to herein.

In general, oligonucleotides may be single-stranded (ss) or double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). Single-stranded DNA is often preferred, as DNA is less susceptible to nuclease degradation than RNA. Similarly, chemical modifications that enhance the specificity or stability of an oligonucleotide or the affinity of an oligonucleotide for a VLP component may be preferred in some applications of the invention. Similar chemical modifications may be made of other nucleic acids used in the practice of the invention. Specific chemical modifications are described elsewhere herein.

Certain types of oligonucleotides are of particular utility in the compositions and methods of the invention, including but not limited to RNAi molecules, antisense oligonucleotides, ribozymes, and aptamers.

(3) Antisense Oligonucleotides

Nucleic acid molecules suitable for use in the practice of the invention include antisense oligonucleotides. In general, antisense oligonucleotides comprise nucleotide sequences sufficient in identity and number to effect specific hybridization with a preselected nucleic acid. Antisense oligonucleotides are generally designed to bind either directly to mRNA transcribed from, or to a selected DNA portion of, a targeted gene, thereby modulating the amount of protein translated from the mRNA or the amount of mRNA transcribed from the gene, respectively. Antisense oligonucleotides may be used as research tools, diagnostic aids, and therapeutic agents.

Antisense oligonucleotides used in accordance with the present invention typically have sequences that are selected to be sufficiently complementary to the target mRNA sequence so that the antisense oligonucleotide forms a stable hybrid with the mRNA and inhibits the translation of the mRNA, often under physiological conditions. Often but not necessarily, the antisense oligonucleotide be 100% complementary to a portion of the target gene. However, the invention also encompasses the production and use of antisense oligonucleotides with a different level of complementarity to the target gene sequence (e.g., in particular instances, antisense oligonucleotides will share at least from about 5% to about 99%, from about 20% to about 99%, from about 30% to about 99%, from about 40% to about 99%, from about 50% to about 99%, from about 60% to about 99%, from about 70% to about 99%, from about 80% to about 99%, from about 85% to about 99%, from about 90% to about 99%, from about 95% to about 99%, from about 70% to about 95%, from about 80% to about 95%, from about 85% to about 95%, from about 90% to about 95%, from about 98% to about 99.9%, or from about 98% to about 99.5% complementary with the target gene sequence).

The amount of sequence homology that an antisense oligonucleotide shares with a target gene will often be determined by the required affinity between the two molecules. Affinity will often be determined by factors such as (1) the particular sequences of the molecules (e.g., the CG-AT ratio), (2) the chemical properties of the antisense oligonucleotide (e.g., chemical properties associated with chemical modifications, and (3) the conditions under which the antisense and target nucleic acids are contacted with each other.

In certain embodiments, antisense oligonucleotide used the practice of the invention will hybridize to an isolated target mRNA under the following conditions: blots are first incubated in prehybridization solution (5×SSC; 25 mM NaPO₄, pH 6.5; 1×Denhardt's solution; and 1% SDS) at 42° C. for at least 2 hours, and then hybridized with radiolabelled cDNA probes or oligonucleotide probes (1×10⁶ cpm/ml of hybridization solution) in hybridization buffer (5×SSC; 25 mM NaPO₄, pH 6.5; 1×Denhardt's solution; 250 μg/ml total RNA; 50% deionized formamide; 1% SDS; and 10% dextran sulfate). Hybridization for 18 hours at 30-42° C. (e.g., 30, 31, 32, 33, 34, 35, 36, 37, 38, 39, 40, 41, or 42° C.) is followed by washing of the filter in 0.1-6×SSC, 0.1% SDS three times at 25-55° C. (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C.). The hybridization temperatures and stringency of the wash will be determined by the percentage of the GC content of the oligonucleotides in accord with the guidelines described by Sambrook et al. (Molecular Cloning: A Laboratory Manual, 2nd edition, 1989, Cold Spring Harbor Laboratory Press, Plainview, N.Y.), including but not limited to Table 11.2 therein.

Representative teachings regarding the synthesis, design, selection and use of antisense oligonucleotides include without limitation U.S. Pat. No. 5,789,573, Antisense Inhibition of ICAM-1, E-Selectin, and CMV IE1/IE2, to Baker et al.; U.S. Pat. No. 6,197,584, Antisense Modulation of CD40 Expression, to Bennett et al.; and Ellington, 1992, Current Protocols in Molecular Biology, 2nd Ed., Ausubel et al., eds., Wiley Interscience, New York, Units 2.11 and 2.12.

(4) Ribozymes

Nucleic acid molecules suitable for use in the present invention also include ribozymes. In general, ribozymes are RNA molecules having enzymatic activities usually associated with cleavage, splicing or ligation of nucleic acid sequences. The typical substrates for ribozymes are RNA molecules, although ribozymes may catalyze reactions in which DNA molecules (or maybe even proteins) serve as substrates. Two distinct regions can be identified in a ribozyme: the binding region which gives the ribozyme its specificity through hybridization to a specific nucleic acid sequence (and possibly also to specific proteins), and a catalytic region which gives the ribozyme the activity of cleavage, ligation or splicing. Ribozymes which are active intracellularly work in cis, catalyzing only a single turnover, and are usually self-modified during the reaction. However, ribozymes can be engineered to act in trans, in a truly catalytic manner, with a turnover greater than one and without being self-modified. Owing to the catalytic nature of the ribozyme, a single ribozyme molecule cleaves many molecules of target RNA and therefore therapeutic activity is achieved in relatively lower concentrations than those required in an antisense treatment (see published PCT patent application WO 96/23569).

Representative teachings regarding the synthesis, design, selection and use of ribozymes include without limitation U.S. Pat. No. 4,987,071 (RNA ribozyme polymerases, dephosphorylases, restriction endoribonucleases and methods) to Cech et al.; and U.S. Pat. No. 5,877,021 (B7-1 Targeted Ribozymes) to Stinchcomb et al.; the disclosures of all of which are incorporated herein by reference in their entireties.

(5) Nucleic Acids for RNAi (RNAi Molecules)

Nucleic acid molecules suitable for use in the present invention also include nucleic acid molecules, particularly oligonucleotides, useful in RNA interference (RNAi). In general, RNAi is one method for analyzing gene function in a sequence-specific manner. For reviews, see Tuschl, Chembiochem. 2:239-245 (2001), and Cullen, Nat. Immunol. 3:597-599 (2002). RNA-mediated gene-specific silencing has been described in a variety of model organisms, including nematodes (Parrish et al., Mol. Cell. 6:1077-1087 (2000); Tabara et al., Cell 99:123-132 (1999)); in plants, i.e., “co-suppression” (Napoli et al., Plant Cell 2:279-289, (1990)) and post-transcriptional or homologous gene silencing (Hamilton et al., Science 286:950-952 (1999); Hamilton et al., EMBO J. 21:4671-4679 (2002)) (PTGS or HGS, respectively) in plants; and in fungi, i.e., “quelling” (Romano et al., Mol. Microbiol. 6:3343-3353 (1992)). Examples of suitable interfering RNAs include siRNAs, shRNAs and stRNAs. As one of ordinary skill will readily appreciate, however, other RNA molecules (e.g., microRNA molecules) having analogous interfering effects are also suitable for use in accordance with this aspect of the invention.

(A) Small Interfering RNA (siRNA)

RNAi is mediated by double-\stranded RNA (dsRNA) molecules that have sequence-specific homology to their “target” RNAs (Caplen et al., Proc. Natl. Acad. Sci. USA 98:9742-9747 (2001)). Biochemical studies in Drosophila cell-free lysates indicates that the mediators of RNA-dependent gene silencing are 21-25 nucleotide “small interfering” RNA duplexes (siRNAs). Accordingly, siRNA molecules are advantageously used in compositions, and methods of the invention. siRNAs may be derived from the processing of dsRNA by an RNase known as DICER (Bernstein et al., Nature 409:363-366, (2001)).

It appears that siRNA duplex products are recruited into a multi-protein siRNA complex termed RISC (RNA Induced Silencing Complex). Without wishing to be bound by any particular theory, it is believed that a RISC is guided to a target mRNA, where the siRNA duplex interacts sequence-specifically to mediate cleavage in a catalytic fashion (Bernstein et al., Nature 409:363-366, 2001; Boutla et al., Curr. Biol. 11:1776-1780 (2001)).

RNAi has been used to analyze gene function and to identify essential genes in mammalian cells (Elbashir et al., Methods 26:199-213 (2002); Harborth et al., J. Cell. Sci. 114:4557-4565 (2001)), including by way of non-limiting example neurons (Krichevsky et al., Proc. Natl. Acad. Sci. USA 99:11926-11929 (2002)). RNAi is also being evaluated for therapeutic modalities, such as inhibiting or block the infection, replication and/or growth of viruses, including without limitation poliovirus (Gitlin et al., Nature 418:379-380 (2002)) and HIV (Capodici et al., J. Immunol. 169:5196-5201 (2002)), and reducing expression of oncogenes (e.g., the bcr-abl gene; Scherr et al., Blood 101:1566-1569 (2003)). RNAi has been used to modulate gene expression in mammalian (mouse) and amphibian (Xenopus) embryos (respectively, Calegari et al., Proc. Natl. Acad. Sci. USA 99:14236-14240 (2002); and Zhou, et al., Nucleic Acids Res. 30:1664-1669 (2002)), and in postnatal mice (Lewis et al., Nat. Genet. 32:107-108 (2002)), and to reduce trangsene expression in adult transgenic mice (McCaffrey et al., Nature 418:38-39 (2002)). Methods have been described for determining the efficacy and specificity of siRNAs in cell culture and in vivo (see, e.g., Bertrand et al., Biochem. Biophys. Res. Commun. 296:1000-1004 (2002); Lassus et al., Sci. STKE 2002 (147):PL13 (2002); and Leirdal et al., Biochem. Biophys. Res. Commun. 295:744-748 (2002)).

Molecules that mediate RNAi, including without limitation siRNA, can be produced in vitro by chemical synthesis (Hohjoh, FEBS Lett. 521:195-199 (2002)), hydrolysis of dsRNA (Yang et al., Proc. Natl. Acad. Sci. USA 99:9942-9947 (2002)), by in vitro transcription with T7 RNA polymerase (Donzeet et al., Nucleic Acids Res. 30:e46, 2002; Yu et al., Proc. Natl. Acad. Sci. USA 99:6047-6052 (2002)), and by hydrolysis of double-stranded RNA using a nuclease such as E. coli RNase III (Yang et al., Proc. Natl. Acad. Sci. USA 99:9942-9947 (2002)).

References regarding siRNA: Bernstein et al., Nature 409:363-366 (2001); Boutla et al., Curr. Biol. 11:1776-1780 (2001); Cullen, Nat. Immunol. 3:597-599 (2002); Caplen et al., Proc. Natl. Acad. Sci. USA 98:9742-9747 (2001); Hamilton et al., Science 286:950-952, (1999); Nagase et al., DNA Res. 6:63-70 (1999); Napoli et al., Plant Cell 2:279-289 (1990); Nicholson et al., Mamm. Genome 13:67-73 (2002); Parrish et al., Mol. Cell. 6:1077-1087 (2000); Romano et al., Mol Microbiol 6:3343-3353 (1992); Tabara et al., Cell 99:123-132 (1999); and Tuschl, Chembiochem. 2:239-245 (2001).

In many instances, siRNAs will be of a length in the range of from about 15 to about 80 nucleotides, from about 15 to about 70 nucleotides, from about 15 to about 60 nucleotides, from about 15 to about 50 nucleotides, from about 15 to about 40 nucleotides, from about 15 to about 35 nucleotides, from about 15 to about 30 nucleotides, from about 15 to about 27 nucleotides, from about 15 to about 26 nucleotides, from about 15 to about 25 nucleotides, from about 15 to about 24 nucleotides, from about 15 to about 23 nucleotides, from about 15 to about 22 nucleotides, from about 18 to about 30 nucleotides, from about 18 to about 27 nucleotides, from about 18 to about 25 nucleotides, from about 20 to about 30 nucleotides, from about 20 to about 28 nucleotides, from about 20 to about 27 nucleotides, from about 20 to about 26 nucleotides, from about 20 to about 25 nucleotides, from about 22 to about 30 nucleotides, from about 22 to about 28 nucleotides, from about 22 to about 27 nucleotides, from about 22 to about 26 nucleotides, from about 22 to about 25 nucleotides, from about 23 to about 30 nucleotides, from about 23 to about 26 nucleotides, from about 23 to about 25 nucleotides, from about 24 to about 30 nucleotides, from about 24 to about 28 nucleotides, from about 24 to about 27 nucleotides, or from about 24 to about 26 nucleotides. In some instances, the siRNA will be of 22, 23, 24, 25, 26, 27, 28, 29, or 30 nucleotides in length.

An siRNA molecule which may be used in the practice of the invention is STEALTH™, available from Invitrogen Corp, Carlsbad, Calif. Additional siRNA molecules which may be used in the practice of the invention are described in U.S. Patent Publication 2006/0009409 A1, the entire disclosure of which is incorporated herein by reference.

siRNA molecules, as well as other nucleic acid molecules, used in the practice of the invention may be blunt ended or have overhangs.

An “overhang” is a relatively short single-stranded nucleotide sequence on the 5′ or 3′ end of a double-stranded oligonucleotide molecule (also referred to as an “extension,” “protruding end,” or “sticky end”).

In some embodiments, with siRNA molecules, as well as other nucleic acid molecules used in the practice of the invention, the length of the sense strand can be 29, 28, 27, 26, 25, 24, 23, 22, 21, 20, 19, or 18 nucleotides. Similarly, the length of the antisense strand can be 29, 28, 27, 26, 25, 24, 23, 22, 21, 20, 19, or 18 nucleotides. Further, when a double-stranded nucleic acid molecule is formed from such sense and antisense molecules, the resulting duplex may have blunt ends or overhangs of 0, 1, 2, 3, 4, 5, 6, 7, 8, 9, 10, 11, 12, 13, or 14 nucleotides on one end or independently on each end. Further, double stranded nucleic acid molecules of the invention may be composed of a sense strand and an antisense strand wherein these strands are of lengths described above, and are of the same or different lengths, but share only 10, 11, 12, 13, 14, 15, 16, 17, 18, 19, or 20 nucleotides of sequence complementarity. By way of illustration, in a situation where the sense strand is 20 nucleotides in length and the antisense is 25 nucleotides in length and the two strands share only 15 nucleotides of sequence complementarity, a double stranded nucleic acid molecules may be formed with a 10 nucleotide overhang on one end and a 5 nucleotide overhang on the other end.

siRNA molecules which can be used in the practice of the invention include STEALTH™ RNAs which may be obtained from Invitrogen Corporation (Carlsbad, Calif.). STEALTH™ RNAs are often synthesized based upon nucleotide sequence information provided by purchasers. In particular instances, purchasers may provide the nucleotide sequence of an RNA transcript for which “knockdown” is desired and Invitrogen Corporation then selects suitable STEALTH™ RNA for the particular application or purchasers may provide the actual sequence of the STEALTH™ RNAs to be used in the “knockdown” process. Typically, in the second instance, the nucleotide sequences provided by purchasers are between 20 and 30 nucleotides in length.

(B) Short Hairpin RNAs (shRNAs)

Paddison et al. (Genes & Dev. 16:948-958 (2002)) have used small RNA molecules folded into hairpins as a means to effect RNAi. Accordingly, such short hairpin RNA (shRNA) molecules are also advantageously used in the methods and compositions of the invention. The length of the stem and loop of functional shRNAs varies; stem lengths can range anywhere from about 25 to about 30 nucleotides, and loop size can range between 4 to about 25 nucleotides without affecting silencing activity. Other stem/loop lengths described herein may also be used. While not wishing to be bound by any particular theory, it is believed that these shRNAs resemble the dsRNA products of the Dicer RNase and, in any event, have the same capacity for inhibiting expression of a specific gene.

Nucleic acid molecules associated with VLPs may either encode shRNAs or may be shRNAs. In any event, in many instances, shRNA molecules will be expressed using an RNA polymerase III promoter. Of course, shRNA molecules may also be made by other methods such as chemical synthesis. Thus, the invention includes the production of shRNA molecule, association of these shRNA molecules with VLPs, and delivery of the shRNA molecules to a cell via association with VLPs. Various aspect of shRNA molecules which may be used in conjunction with the invention include those described elsewhere herein.

In many instances, shRNA molecules, when generated using a vectors (e.g., an expression vector), will be transcribed from nucleic acid which is operably connected to an RNA polymerase III promoter (e.g., a U6 or H1 promoter).

Transcriptional termination by RNA polymerase III is known to occur at runs of four consecutive T residues in the DNA template (Tazi, J. et al., Mol. Cell. Biol. 13:1641-50 (1993); and Booth & Pugh, J. Biol. Chem. 272:984-91 (1997)), providing one mechanism to end a shRNA transcript at a specific sequence. In addition, previous studies have demonstrated that the RNA polymerase III based expression vectors could be used for the synthesis of short RNA molecules in mammalian cells (Noonberg et al., Nucleic Acids Res 22:2830-2836 (1994); and Good et al., Gene Ther 4:45-54 (1997)). While most genes transcribed by RNA polymerase III require cis-acting regulatory elements within their transcribed regions, the regulatory elements for the U6 small nuclear RNA gene are contained in a discrete promoter located 5′ to the U6 transcript (Reddy, J. Biol. Chem. 263:15980-15984 (1988)). Using an expression vector with a mouse U6 promoter, it has been shown that both hairpin shRNAs expressed in cells can inhibit gene expression.

Nucleic acid molecules which may be used in the practice of the invention include those generated by the BLOCK-IT™ line of products available from Invitrogen Corp. (Carlsbad, Calif.). Examples of such products include those entitled BLOCK-IT™ Inducible H1 RNAi Entry Vector Kit (catalog no. K4920-00), BLOCK-IT™ Inducible H1 Lentiviral RNAi System (catalog no. K4925-00), and BLOCK-IT™ U6 RNAi Entry Vector (catalog no. K4945-00).

(C) MicroRNAs

Another group of small RNAs suitable for use in the composition and methods of the invention are microRNAs. MicroRNAs (mRNAs) are short non-coding RNAs that play a role in the control of gene expression. It has been estimated that as much as 1% of the human genome may encode mRNA (Lim et al., Science 299:1540 (2003).

MicroRNA molecules are molecules which are structurally similar to shRNA molecules but, typically, contain one or more (e.g., one, two, three, four, five, six, etc.) mismatches or insertion/deletions in their regions of sequence complementary. At least some microRNA molecules are transcribed as polycistrons of about 400, which are then processed to RNA molecules of about 70 nucleotides. These double stranded 70 mers are then are processed again, presumably by the enzyme Dicer, to two RNA molecules which are about 22 nucleotides in length and often have one or more (e.g., one, two, three, four, five, etc.) internal mismatches in their regions of sequence complementarity. Lee et al., EMBO 21:4663-4670 (2002). Thus, the invention also includes, for example, methods and compositions comprising microRNAs.

MicroRNA may be expressed using RNA polymerase II promoters, which offers several advantages over RNA polymerase III expression systems. First, the technology for expression from RNA polymerase II promoters to achieve tissue specific or inducible/repressible expression is well developed. Second, under some conditions, RNA polymerase II based hairpin RNA molecule production may be more suitable than RNA polymerase III hairpin RNA molecule production for retroviral delivery, since retroviruses contain RNA polymerase II promoters. Third, RNA polymerase II does not terminate at runs of four thymidines in a template sequence, which allows for greater flexibility in RNA design. For example, in some embodiments it may be desirable to include 3 or more consecutive U nucleotides within an RNA molecule. Such an RNA may be difficult to synthesize using an RNA polymerase III expression system, because the consecutive Ts/Us would tend to cause termination of transcription.

While not being bound by theory, the current model for the maturation of mammalian microRNAs is considered to be essentially as follows (explained in more detail in PCT Publication WO 2006/092738. Gene coding for microRNAs are typically transcribed resulting in the production of an microRNA precursor known as the pre-microRNA. The pre-microRNA can be part of a polycistronic RNA comprising multiple pre-microRNAs. Pre-microRNAs typically form a hairpin with a stem and loop where the stem may contain one or more mismatched bases.

The hairpin structure of the pre-microRNA is believed to be recognized by Drosha, which is an RNase III endonuclease. Drosha is believed to recognize terminal loops in the pre-microRNA and cleave approximately two helical turns into the stern to produce a 60-70 nucleotide precursor known as the pre-microRNA. Drosha is believed to cleave the pre-microRNA with a staggered cut typical of RNase III endonucleases resulting in a pre-microRNA stem loop with a 5′ phosphate and about a two nucleotide 3′ overhang. About one helical turn of the stem (about 10 nucleotides) extending beyond the Drosha cleavage site may be required for efficient processing. It is believed that the pre-microRNA is then actively transported from the nucleus to the cytoplasm by Ran-GTP and the export receptor Ex-portin-5.

Further, the pre-microRNA is believed to also be recognized by Dicer, which is also an RNase III endonuclease. Dicer is believed to recognize the double-stranded stem of the pre-microRNA. Dicer may also recognize the 5′ phosphate and 3′ overhang at the base of the stem loop and may cleave off the terminal loop two helical turns away from the base of the stem loop leaving an additional 5′ phosphate and about a two nucleotide 3′ overhang. The resulting shRNA-like duplex, which may contain one or more mismatches, forms the mature microRNA.

The microRNA is believed to eventually be incorporated as a single-stranded RNA into a ribonucleoprotein complex known as the RNA-induced silencing complex or RISC. The RISC is believed to identify target nucleic acids, for which cleavage occurs, based on high levels of complementarity between the microRNA and the mRNA

While rules for the design of efficient microRNAs are still being worked out, several studies have reviewed the base-pairing requirement between microRNA and its mRNA target for achieving efficient inhibition of translation (reviewed in Bartel, Cell 116:281-297 (2004)). In any event, the mismatches of the stem strands may lead to a population of different hairpin structures. Variability in the stem structures may also lead to variability in the products of cleavage by Drosha and Dicer. Thus, in many embodiments, mixed populations of microRNAs may be employed. These populations may vary from one another, for example, in (1) the sequences of the stems and/or loops and/or (2) the number of mismatches in the stem region.

MicroRNAs used in the practice of the invention, may be synthesized by as a polycistron. Along these lines, microRNAs may be included within introns of genes or may be transcribed along with one or more other microRNAs as part of the same transcript.

A initial transcript containing a microRNA used in the practice of the invention may be from about 70 to about 5,000, from about 80 to about 5,000, from about 100 to about 5,000, from about 140 to about 5,000, from about 70 to about 200, from about 70 to about 400, from about 80 to about 200, from about 90 to about 200, from about 100 to about 200, from about 110 to about 200, from about 70 to about 400, from about 70 to about 600, from about 70 to about 800, from about 70 to about 1,000, from about 70 to about 2,500, or from about 100 to about 2,000 nucleotides in length.

VLPs may be used to deliver microRNAs which are at any stage of processing. Thus, VLPs may be associated with pre-microRNAs, microRNAs, of microRNAs which have been processed to the point where there are composed of two separate nucleic acid stranded of less than about 30 nucleotides each in length.

A number of microRNA products may be used in or adapted for use with the invention. Examples of such products include the “BLOCK-IT™ Pol II miR RNAi Expression Vectors” available from Invitrogen Corp., Carlsbad, Calif. (see, e.g., cat. nos. K4935-00, K4936-00, K4937-00, K4938-00, V49350-00, V49351-00, and V49352-00).

(6) Oligonucleotide Synthesis

The oligonucleotides, as well as many other nucleic acid molecules, used in accordance with the invention can be conveniently and routinely made through the well-known technique of solid-phase synthesis. Equipment for such synthesis is sold by several vendors including, for example, Applied Biosystems (Foster City, Calif.). Other methods for such synthesis that are known in the art may additionally or alternatively be employed. It is well known to use similar techniques to prepare oligonucleotides such as the phosphorothioates and alkylated derivatives. By way of non-limiting example, see, e.g., U.S. Pat. No. 4,517,338 (Multiple reactor system and method for polynucleotide synthesis) to Urdea et al., and U.S. Pat. No. 4,458,066 (Process for preparing polynucleotides) to Caruthers et al.; Lyer et al., Modified oligonucleotides—synthesis, properties and applications. Curr Opin Mol. Ther. 1:344-358, 1999; Verma et al., Modified oligonucleotides: synthesis and strategy for users. Annu Rev Biochem. 67:99-134, 1998; Pfleiderer et al., Recent progress in oligonucleotide synthesis. Acta Biochim Pol. 43:37-44, 1996; Warren et al., Principles and methods for the analysis and purification of synthetic deoxyribonucleotides by high-performance liquid chromatography. Mol Biotechnol. 4:179-199, 1995; Sproat, Chemistry and applications of oligonucleotide analogues. J Biotechnol. 41:221-238, 1995; De Mesmaeker et al., Backbone modifications in oligonucleotides and peptide nucleic acid systems. Curr. Opin. Struct. Biol., 5:343-355, 1995; Charubala et al., Chemical synthesis of 2′,5′-oligoadenylate analogues. Prog. Mol. Subcell. Biol., 14:114-138, 1994; Sonveaux, Protecting groups in oligonucleotide synthesis. Methods Mol Biol. 26:1-71, 1994; Goodchild, Conjugates of oligonucleotides and modified oligonucleotides: a review of their synthesis and properties. Bioconjug Chem. 1:165-187, 1990; Thuong et al., Chemical synthesis of natural and modified oligodeoxynucleotides. Biochimie 67:673-684, 1985; Itakura et al., Synthesis and use of synthetic oligonucleotides. Annu Rev Biochem. 53:323-356, 1984; Caruthers et al., Deoxyoligonucleotide synthesis via the phosphoramidite method. Gene Amplif Anal. 3:1-26, 1983; Ohtsuka et al., Recent developments in the chemical synthesis of polynucleotides. Nucleic Acids Res. 10:6553-6560, 1982; and Kossel, Recent advances in polynucleotide synthesis. Fortschr Chem Org Naturst. 32:297-508, 1975.

(7) Chemical Modifications of Nucleic Acids

In certain embodiments, particularly those involving synthetic nucleic acids, oligonucleotides used in accordance with the present invention may comprise one or more chemical modifications. By way of non-limiting example, Braasch et al. (Biochemistry 42:7967-75, 2003) report that RNAi molecules at least tolerate, and may be enhanced by, phosphorothioate linkages and/or the incorporation of 2′-deoxy-2′-fluorouridine. Chemical modifications include with neither limitation nor exclusivity base modifications, sugar modifications, and backbone modifications. In addition, a variety of molecules, including by way of non-limiting example fluorophores and other detectable moieties, can be conjugated to the oligonucleotides or incorporated therein during synthesis. Other suitable modifications include but are not limited to base modifications, sugar modifications, backbone modifications, and the like.

(A) Base Modifications

In certain embodiments, the oligonucleotides used in the present invention can comprise one or more base modifications. For example, the base residues in aptamers may be other than naturally occurring bases (e.g., A, G, C, T, U, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine (and derivatives thereof), N6-isopentenyladenine, 1-methyladenine, 1-methylpseudouracil, 1-methylguanine, 1-methylinosine, 2,2-dimethylguanine, 2-methyladenine, 2-methylguanine, 7-methylguanine, 3-methylcytosine, 5-methylcytosine (5MC), N6-methyladenine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in oligonucleotides and other nucleic acids.

(B) Sugar Modifications

The oligonucleotides used in the present invention can also (or alternatively) comprise one or more sugar modifications. For example, the sugar residues in oligonucleotides and other nucleic acids may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-alkyl, 2′-S-allyl, 2′-fluoro-, 2′-halo, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside, ethyl riboside or propylriboside.

Sugar moieties include natural, unmodified sugars, e.g., monosaccharides (such as pentoses, e.g., ribose, deoxyribose), modified sugars and sugar analogs. Possible modifications of nucleomonomers, particularly of a sugar moiety, include, for example, replacement of one or more of the hydroxyl groups with a halogen, a heteroatom, an aliphatic group, or the functionalization of the hydroxyl group as an ether, an amine, a thiol, or the like. One particularly useful group of modified nucleomonomers are 2′-O-methyl nucleotides, especially when the 2′-O-methyl nucleotides are used as nucleomonomers in the ends of the oligomers. Such 2′O-methyl nucleotides may be referred to as “methylated,” and the corresponding nucleotides may be made from unmethylated nucleotides followed by alkylation or directly from methylated nucleotide reagents. Modified nucleomonomers may be used in combination with unmodified nucleomonomers. For example, an oligonucleotide of the invention may contain both methylated and unmethylated nucleomonomers.

(C) Backbone Modifications

The oligonucleotides used in the present invention can also (or alternatively) comprise one or more backbone modifications. For example, chemically modified backbones of oligonucleotides and other nucleic acids include, by way of non-limiting example, phosphorothioates, chiral phosphorothioates, phosphorodithioates, phosphotriesters, aminoalkylphos-photriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotri-esters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Chemically modified backbones that do not contain a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages, including without limitation morpholino linkages; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; and amide backbones.

(D) Exemplary Chemical Modifications

Some exemplary modified nucleomonomers include sugar- or backbone-modified ribonucleotides. Modified ribonucleotides may contain a nonnaturally occurring base (instead of a naturally occurring base) such as uridines or cytidines modified at the 5-position, e.g., 5-(2-amino)propyl uridine and 5-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides may have the 2′-OH group replaced by a H, alxoxy (or OR), R or alkyl, halogen, SH, SR, amino (such as NH₂, NHR, NR_{2,}), or CN group, wherein R is lower alkyl, alkenyl, or alkynyl.

Modified ribonucleotides may also have the phosphoester group connecting to adjacent ribonucleotides replaced by a modified group, e.g., of phosphothioate group. More generally, the various nucleotide modifications may be combined.

In one embodiment, sense oligomers may have 2′ modifications on the ends (1 on each end, 2 on each end, 3 on each end, and 4 on each end, and so on; as well as 1 on one end, 2 on one end, 3 on one end, and 4 on one end, and so on; and even unbalanced combinations such as 1 on one end and 2 on the other end, and so on). Likewise, the antisense strand may have 2′ modifications on the ends (1 on each end, 2 on each end, 3 on each end, and 4 on each end, and so on; as well as 1 on one end, 2 on one end, 3 on one end, and 4 on one end, and so on; and even unbalanced combinations such as 1 on one end and 2 on the other end, and so on). In preferred aspects, such 2′-modifications are in the sense RNA strand or the sequences other than the antisense strand.

To further maximize endo- and exonuclease resistance, in addition to the use of 2′ modified nucleomonomers in the ends, inter-nucleomonomer linkages other than phosphodiesters may be used. For example, such end blocks may be used alone or in conjunction with phosphothioate linkages between the 2′-O-methyl linkages. Preferred 2′-modified nucleomonomers are 2′-modified C and U bases.

Although the antisense strand may be substantially identical to at least a portion of the target gene (or genes), at least with respect to the base pairing properties, the sequence need not be perfectly identical to be useful, e.g., to inhibit expression of a target gene's phenotype. Generally, higher homology can be used to compensate for the use of a shorter antisense gene. In some cases, the antisense strand generally will be substantially identical (although in antisense orientation) to the target gene.

One particular example of a composition of the invention has end-blocks on both ends of a sense oligonucleotide and only the 3′ end of an antisense oligonucleotide. Without wishing to be bound by theory, the inventors believe that a 2′-O-modified sense strand works less well than unmodified because it is not efficiently unwound. Accordingly, another embodiment of the invention includes duplexes in which nucleomonomer-nucleomonomer mismatches are present in a sense 2′-O-methyl strand (and are thought to be easier to unwind).

Accordingly, for a given first oligonucleotide strand, a number of complementary second oligonucleotide strands are permitted according to the invention. For example, in the following Tables, a targeted and a non-targeted oligonucleotide are illustrated with several possible complementary oligonucleotides. The individual nucleotides may be 2′-OH RNA nucleotides (R) or the corresponding 2′-O-methyl nucleotides (M), and the oligonucleotides themselves may contain mismatched nucleotides (lower case letters).

### Targeted Oligonucleotide:

| | | |
|---|---|---|
| First Strand: | CCCUUCUGUCUUGAACAUGAG | (SEQ ID NO: 2 ) |
| Second Strand: | CTgATGTTCAAGACAGAAcGG | (SEQ ID NO: 3 ) |
| (methyl | MMMMMMMMMMMMMMMMMMMMM | |
| groups →) | | |
| | CTgATGTTCAAGACAGAAcGG | (SEQ ID NO: 4) |
| | RRRRRRRRRRRRRRRRRRRDD | |
| | CTCAUGUUCAAGACAGAAGGG | (SEQ ID NO: 5) |
| | RRRRRRMMMMMMMMMRRRRRR | |
| | CTCAUGUUCAAGACAGAAGGG | (SEQ ID NO: 6) |
| | MMMMMMRRRRRRRRRMMMMMM | |
| | CTCAUGUUCAAGACAGAAGGG | (SEQ ID NO: 7) |
| | RMRMRMRMRMRMRMRMRMRMR | |

### Non-Targeted Oligonucleotide:

| | | |
|---|---|---|
| First Strand: | GAGTACAAGTTCTGTCTTCCC | (SEQ ID NO: 8) |
| Second Strand: | GGcAAGACAGAACTTGTAgTC | (SEQ ID NO: 9) |
| (methyl | MMMMMMMMMMMMMMMMMMMMM | |
| groups →) | | |
| | GGGAAGACAGAACTTGTACTC | (SEQ ID NO: 10) |
| | RRRRRRMMMMMMMMMRRRRRR | |
| | GGGAAGACAGAACTTGTACTC | (SEQ ID NO: 11) |
| | MMMMMMRRRRRRRRRMMMMMM | |
| | GGGAAGACAGAACTTGTACTC | (SEQ ID NO: 12) |
| | RMRMRMRMRMRMRMRMRMRMR | |

Another example of further modifications that may be used in conjunction with 2′-O-methyl nucleomonomers are modification of the sugar residues themselves, for example alternating modified and unmodified sugars, particularly in the sense strand.

The invention further includes double stranded nucleic acid molecules (e.g., RNA molecules) which have structures defined by the following formula:

| | |
|---|---|
| First Strand | X₁₅₋₃₀ |
| Second Strand | A₀₋₂₅X₀₋₂₅B₀₋₂₅ |

In the formula set out above, X, A, and B are nucleotides (e.g., A, G, C, U, etc.). Also, either of the first strand or the second strand may be a sense strand. As a results, either of the first strand or the second strand may be an antisense strand. Further, X is typically a nucleotide which has no modifications on the base or sugar. Further, A and/or B are nucleotides which may independently contain one or more base or sugar modifications. These modifications may be any modifications known in the art or described elsewhere herein. Examples of sugar modifications include ribose modifications at the 2′ position such as 2′-O-propyl (P), 2′-O-methyl (M), 2′-O-ethyl (E), and 2′-fluoro (F). Generic examples of nucleic acid molecules of the invention include those with the following:

XXXXXXXXXXXXXXXXXXXX
AXXXXXXXXXXXXXXXXXXB
XXXXXXXXXXXXXXXXXXXX
AAXXXXXXXXXXXXXXXXBB
XXXXXXXXXXXXXXXXXXXX
AAAXXXXXXXXXXXXXXBBB
XXXXXXXXXXXXXXXXXXXX
AAAAXXXXXXXXXXXXBBBB
XXXXXXXXXXXXXXXXXXXX
AAAAXXXXXXXXXXXXXXBB
XXXXXXXXXXXXXXXXXXXX
AAXXXXXXXXXXXXXBBBBB
XXXXXXXXXXXXXXXXXXXX
AAAAAAAAAAAAAAAAAAAA
XXXXXXXXXXXXXXXXXXXX
AAAAAAAXXXBBBBBBBBBB

Examples of nucleic acid molecules of the invention which contain specific modifications include those with the following modifications, in which X represents an unmodified nucleotide, P represents 2′-O-propyl, M represents 2′-O-methyl, E represents 2′-O-ethyl, and F represents 2′-fluoro:

XXXXXXXXXXXXXXXXXXXXXXXXX
PPMMXXXXXXXXXXXXXXXXEEMMM
XXXXXXXXXXXXXXXXXXXXXXXXX
EEEEXXXXXXXXXXXXXXXXEEMMM
XXXXXXXXXXXXXXXXXXXXXXXXX
PPEEXXXXXXXXXXXXXXXXEEMMM
XXXXXXXXXXXXXXXXXXXXXXXXX
EEEEEXXXXXXXXXXXXXXXEEEEE
XXXXXXXXXXXXXXXXXXXXXXXXX
PPPPPPPXXXXXXXXXXXPPPPPPP
XXXXXXXXXXXXXXXXXXXXXXXXX
FFPPPXXXXXXXXXXXXXXXPPPFF
XXXXXXXXXXXXXXXXXXXXXXXXX
MPPPPPPPPPPPPPPPPXXXPPPPM
XXXXXXXXXXXXXXXXXXXXXXXXX
FFFFFXXXXXXXXXXXXXXXFFFFF
XXXXXXXXXXXXXXXXXXXXXXXXX
PEEPEEMPXXXXXXXXXPMEEPEEP
XXXXXXXXXXXXXXXXXXXXXXXXX
MEXXXXXXXXXXXXXXMMMMMMMMM
XXXXXXXXXXXXXXXXXXXXXXXXX
MXXXXXXXXXXXXXXXMMMMMMMMM
XXXXXXXXXXXXXXXXXXXXXXXXX
EEXXXXXXXXXXXXXXXEEEEEEEE

In some embodiments, the length of the sense strand can be 29, 28, 27, 26, 25, 24, 23, 22, 21, 20, 19, or 18 nucleotides. Similarly, the length of the antisense strand can be 29, 28, 27, 26, 25, 24, 23, 22, 21, 20, 19, or 18 nucleotides. Further, when a double-stranded nucleic acid molecule is formed from such sense and antisense molecules, the resulting duplex may have blunt ends or overhangs of 0, 1, 2, 3, 4, 5, 6, 7, 8, 9, 10, 11, 12, 13, or 14 nucleotides on one end or independently on each end. Further, double stranded nucleic acid molecules of the invention may be composed of a sense strand and an antisense strand wherein these strands are of lengths described above, and are of the same or different lengths, but share only 10, 11, 12, 13, 14, 15, 16, 17, 18, 19, or 20 nucleotides of sequence complementarity. By way of illustration, in a situation where the sense strand is 20 nucleotides in length and the antisense is 25 nucleotides in length and the two strands share only 15 nucleotides of sequence complementarity, a double stranded nucleic acid molecules may be formed with a 10 nucleotide overhang on one end and a 5 nucleotide overhang on the other end.

Double-stranded oligonucleotides of the invention include STEALTH™ RNAs which may be obtained from Invitrogen Corporation (Carlsbad, Calif.). STEALTH™ RNAs are often synthesized based upon nucleotide sequence information provided by purchasers. In particular instances, purchasers may provide the nucleotide sequence of an RNA transcript for which “knockdown” is desired and Invitrogen Corporation (Carlsbad, Calif.) then selects suitable STEALTH™ RNA for the particular application or purchasers may provide the actual sequence of the STEALTH™ RNAs to be used in the “knockdown” process. Typically, in the second instance, the nucleotide sequences provided by purchasers are between 20 and 30 nucleotides in length. A more detailed description of business method aspects of the invention is set out elsewhere herein. However, these business methods typically include, in part, providing STEALTH™ RNA, as well as protocols and additional reagents and compounds for purchasers to use the purchased STEALTH™ RNA for knocking down gene expression.

(B) Polypeptides

Methods and compositions of the invention may also be used to deliver one or more polypeptides (e.g., heterologous polypeptides) to cells. Thus, the invention provide methods for preparing VLPs which are associated with one or more polypeptide, as well as methods for preparing such VLPs, methods for introducing polypeptides into cells, compositions comprising VLPs which are associated with one or more polypeptides, and components used to prepare such VLPs.

In many instances, the one or more polypeptides referred to above will be a heterologous polypeptide, such as a polypeptide which is not normally associated with a VLP or a polypeptide which is not normally associated with a cell used to produce the particular VLPs.

Characteristics of the polypeptides used in conjunction with the invention may vary greatly but include the following: size, activity, charge, hydrophobicity/hydrophilicity, secondary structure, tertiary structure, quaternary structure, composite structure, ligand binding properties, covalent linkage to a another compound (e.g., covalent linkage to a non-polypeptide or to a polypeptide by a linkage other than a peptide bond, such as via a sulfhydryl group of a cysteine residue or a hydroxyl group of a serine or threonine residue.)

The characteristics of the polypeptide will vary with a number of factors including the particular reason why one wishes to have it associated with a VLP and how the polypeptide is associated with the VLP. As an example, when a VLP is intended to be included with the capsid of a VLP, there may be limitations on the permissible size and charge of the polypeptide.

Typically, nucleic acid will also be present in a VLP. Thus, polypeptides with either a net positive charge of with one or more regions of net positive charge will generally be more likely to be included in VLPs. Similarly, polypeptides with affinity for nucleic acids will be more likely to be included and/or will be included with higher frequency within VLPs. Thus, polypeptides which interact with nucleic acids may be delivered by methods of the invention. Examples of such polypeptides include gyrases, topoisomerases, recombinases, proteins with zinc finger domains, DNA repair proteins (e.g., RecA and mis-match repair proteins such as MLH1 and PMS2), histones, protamines, single-stranded binding proteins, viral proteins (e.g., SV40 large T antigen), expression regulators (e.g., p53), polymerases (e.g., DNA polymerases, RNA polymerases). Polypeptide related to those above but which have been altered to change their activity may also be used in the invention. One example of such a polypeptide is PMS2. hPMS2-134, which carries a truncation mutation at codon 134 is an example of a dominant negative allele of a mismatch repair gene. The mutation causes the product of this gene to abnormally terminate at the position of the 134th amino acid, resulting in a shortened polypeptide containing the N-terminal 133 amino acids. Such a mutation causes an increase in the rate of mutations which accumulate in cells after DNA replication. Expression of a dominant negative allele of a mismatch repair gene results in impairment of mismatch repair activity, even in the presence of the wild-type allele. This system is described in U.S. Pat. No. 6,825,038, the entire disclosure of which is incorporated herein by reference. Thus, the invention includes methods and compositions for introducing polypeptides which confer specific phenotypes onto cells.

Polypeptides may also be associated with VLPs via affinity to a VLP protein such as a capsid protein. For example, the polypeptide may be covalently or non-covalently attached to a VLP protein. For covalent attached, a VLP protein and the polypeptide may be expressed as a fusion protein. Alternatively, a polypeptide may be covalently attached to a VLP protein via a covalent bond other than a peptide bond or by a peptide bond which generated after production of the VLP protein and the polypeptide.

Any number of attractions may be used to connect a polypeptide to a VLP protein non-covalently. As an example, cyclophilin A has been shown to interact with lentiviral capsid proteins (see, e.g., Lin and Emerma, Retrovirology, 3:70-82 (2006)). Thus, polypeptides used in the practice of the invention may be naturally occurring polypeptide or polypeptides which contain one or more regions which have affinity for a VLP protein. Along these lines, the invention include compositions and methods which employ fusion protein, wherein the fusion protein contains at least one region with affinity for a VLP protein and another region which confers upon the fusion protein an activity which is sought to be delivered to a cell.

Modified VLP proteins may also be used to deliver compounds to cells. For example, a VLP protein may be modified to introduce an affinity for a compound. As more specific example, a compound may be conjugated to biotin and a VLP protein may be expressed with suitable amino acid sequences of Streptavidin to allow for connection of the compound to the VLP protein.

Polypeptides may also be associated with VLPs by connection to the envelope, when present. When a polypeptide is associated with a VLP via the envelope, the polypeptide may be embedded in the envelope or by binding to a molecule which is present in the envelope.

In one embodiment, the invention include methods for preparing VLPs which are associated with a compound (e.g., a polypeptide with at least one hydrophobic region) through an envelope. In specific embodiments, cells are prepared which have the compound associated with the envelope followed by the formation of enveloped VLPs. In many instances, these VLPs will acquire the compound when the envelope forms. Further, also in many instances, the amount of compound associated with the VLPs will relate to the amount of compound present in the cell's enveloped.

Of course, there are any number of additional ways to associate compounds with VLP envelopes. One method is to produce a VLP containing a membrane bound protein with a Streptavidin region followed by connection of a compound which contains a biotin moiety. In many instances, such compounds would be present on the outside of the envelope.

Polypeptides used in the practice of the invention may contain any number of amino acids including from about 10 to about 10,000, from about 50 to about 10,000, from about 100 to about 10,000, from about 200 to about 10,000, from about 4000 to about 10,000, from about 10 to about 50, from about 10 to about 100, from about 10 to about 200, from about 10 to about 400, from about 10 to about 500, from about 15 to about 25, from about 15 to about 50, from about 15 to about 100, from about 15 to about 200, from about 15 to about 500, from about 20 to about 30, from about 20 to about 50, from about 20 to about 100, from about 20 to about 200, from about 20 to about 400, from about 30 to about 50, from about 30 to about 70, from about 30 to about 100, from about 30 to about 250, from about 40 to about 60, from about 40 to about 80, from about 40 to about 100, from about 40 to about 200, from about 50 to about 150, etc.

(C) Carbohydrates

Carbohydrates are additional examples of compounds which may be used in the practice of the invention. Any number of carbohydrates (e.g., monosaccharides, disaccharide, trisacharides, polysaccharides, etc.) may be delivered to cell by VLPs in the practice of the invention.

Carbohydrates used in the invention may be cyclic or linear and include, for example, aldoses, ketoses, amino sugars, alditols, inositols, aldonic acids, uronic acids, or aldaric acids, or combinations thereof. These carbohydrates may also be a mono-, a di-, or a poly-carbohydrate, such as for example, a disaccharide or polysaccharide. Suitable specific carbohydrates and classes of carbohydrates include for example, arabinose, lyxose, pentose, ribose, xylose, galactose, glucose, hexose, idose, mannose, talose, heptose, glucose, fructose, gluconic acid, sorbitol, lactose, mannitol, methyl-α-glucopyranoside, maltose, isoascorbic acid, ascorbic acid, lactone, sorbose, glucaric acid, erythrose, threose, arabinose, allose, altrose, gulose, idose, talose, erythrulose, ribulose, xylulose, psicose, tagatose, glucuronic acid, gluconic acid, glucaric acid, galacturonic acid, mannuronic acid, glucosamine, galactosamine, sucrose, trehalose or neuraminic acid, or derivatives thereof. Additional carbohydrates include, for example, arabinans, fructans, fucans, galactans, galacturonans, glucans, mannans, xylans (such as, for example, inulin), levan, fucoidan, carrageenan, galactocarolose, pectins, pectic acids, amylose, pullulan, glycogen, amylopectin, cellulose, dextran, pustulan, chitin, agarose, keratin, chondroitin, dermatan, hyaluronic acid, alginic acid, xanthin gum, or starch.

As with polypeptides, carbohydrate compounds may be associated with VLPs in any number of ways and to any number of VLP components. For example, carbohydrates may be connected to a protein which normally resides in the VLP envelope, when present.

(D) Other Compounds

Additional compounds such as drugs (e.g., protein and non-proteins drugs) and labels (e.g., dyes) may be used in the practice of the invention. In many instances such compounds will be bound to other molecules. For example, the invention include methods for delivering to cells nucleic acids which are covalently linked to a fluorescent dye such as fluorescein. Such method allow for detection of delivery events via detection of intracellular fluorescence.

As an example, Kaufmann and Weberskirch, Macromol. Biosci. 6:952-958 (2006) describes the conjugation of doxorubicin-glycine-phenylalanine-leucine-glycine and rhodamine-glycine-phenylalanine-leucine-glycine units to a monodisperse elastin-mimetic protein (EMM) and suggest the use of such drug carriers for cancer therapy. Thus, the invention includes methods for delivering drug-conjugates to cells. In many instances, the drug would be conjugated to a molecule which is associated with a VLP. Further, the invention may be used for cell-type specific delivery of drugs by employing VLPs which will deliver compounds to specific cells. Thus, the invention further includes therapeutic methods employing VLPs to deliver compounds (e.g., drugs) to specific cell-types in an organism.

Examples of drugs which may be used in conjunction with the invention include nucleoside analogues (e.g., acyclovir, gancyclovir, idoxuridine, ribavirin, vidaribine, zidovudine, didanosine and 2′,3′-dideoxycytidine (ddC), amantadine, etc.), antibiotics (e.g., sulphonamides, such as sulanilamide, sulphacarbamide and sulphamethoxydiazine; penicillins, such as 6-aminopenicillanic acid, penicillin G and penicillin V; isoxazoylpenicillins, such as oxacillin, cloxacillin, flucloxacillin; α-substituted benzylpenicillins, such as ampicillin, carbenicillin, pivampicillin and amoxicillin; acylaminopenicillins, such as mezlocillin, azlocillin, piperacillin and apalicillin; tetracyclines, such as tetracycline, chlortetracycline, oxytetracycline, demeclocycline, rolitetracycline, doxycycline and minocycline; chloramphenicols, such as chloramphenicol and thiamphenicol; gyrase inhibitors, such as nalixidic acid, pipemidic acid, norfloxacin, ofloxacin, ciprofloxacin and enoxacin; tuberculosis agents, such as isoniazid; cytokines, such as interleukin 2, interferon α-2a, interferon α-2b, interferon β-1a, interferon β-1b, and interferon γ-1b, etc.

Examples of labels which may be used in conjunction with the invention include fluorescent labels such as 4-acetamido-4′-isothiocyanatostilbene-2-2′-disulfonic acid, 7-amino-4-methylcoumarin (AMC), 7-amino-4-trifluoromethylcoumarin, N-(4-anilino-1-naphthyl) maleimide, 4′,6-diamidino-2-phenylindole (DAPI), 5-(4,6-dichlorotriazin-2-yl) aminofluorescein (DTAF), 4,4′-dilsothiocyanatostilbene-2,2′-disulfonic acid, tetramethylrhodamine isothiocyanate (TRITC), quinolizino fluorescein isothiocyanate (QFITC), dansyl chloride, eosin, isothiocyanate, erythrosin B, fluorescamine, fluorescene, fluorescein derivatives, 4-methylumbelliferone, o-phthaldialdehyde, rhodamine B, rhodamine B derivatives, rhodamine 6G, rhodamine 123, sulforhodamine B, sulforhodamine 101, sulforhodamine 101 acid chloride, etc. Additional labels are described in U.S. Patent Publication No. 2003/0162198, the entire disclosure of which is incorporated herein by reference.

The invention thus includes methods for delivering drugs and labels into cells. In many instances, these methods will be cell type specific. In some instances, the cell-type specificity may be conferred by the VLP components employed.

### Cells for Preparing VLPs

In many instances, VLPs will be prepared using cells (e.g., mammalian cells). The type of cell chosen for preparing VLPs will vary with a number of factors including the type of VLP to be produced and the specific compound to be associated with the VLP. Cells which may be used in the practice of the invention include prokaryotic cells and eukaryotic cells. Exemplary prokaryotic cells include Escherichia coli, Salmonella typhimurium, Staphylococcus aureus, Staphylococcus epidermis, Pseudomonas aeruginosa, and Serratia marcesans, as well as other prokaryotic cells which may be used to produce VLPs or are capable of infection by phage. Exemplary eukaryotic cells include CHO, VERY, BHK, Hela, COS, MDCK, 293, 3T3, WI38, breast cancer cell lines, such as BT483, Hs578T, HTB2, BT20 and T47D, mammary gland cell lines, such as CRL7030 and Hs578Bst, fungal cells, such as yeast cells (e.g., Saccharomyces cerevisiae or Pichia pastoris (ATCC Accession No. 201178)); insect cells such as Drosophila S2 and Spodoptera Sf9 cells. Cell lines which may be used in the practice of the invention also include cells from Invitrogen Corporation (Carlsbad, Calif.) 293FT cells (cat. no. R700-07), 293A cells (cat. no. K4940-00), and One Shot Stbl3 Chemically Competent E. coli (cat. no. C7373-03)

In many instances, it will be necessary to get molecules into cells used to prepare VLPs. Methods used for such purposes will vary with the particular molecules which are sought to be introduced into the cells. For example, transfection reagents may be used to get compounds, nucleic acid molecules which encode compounds, and nucleic acid molecules which encode VLPs components into cells. These cells may then be used to produce VLPs.

Introduction of molecules such as nucleic acids into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see e.g., WO 90/14074; WO 91/16024; WO 91/17424; U.S. Pat. No. 4,897,355; Bergan et al. Nucleic Acids Res. 21:3567 (1993)). Enhanced introduction of molecules can also be mediated by the use of vectors (See e.g., Shi et al., Trends. Genet. 19:9 (2003); Reichhart et al., Genesis, 34:160-4 (2002), Yu et al. 2002. Proc. Natl. Acad. Sci. USA 99:6047 (2002); Sui et al., Proc. Natl. Acad. Sci. USA 99:5515 (2002)) viruses, polyamine or polycation conjugates using compounds such as polylysine, protamine, or N1, N12-bis(ethyl) spermine (see, e.g., Bartzatt, R. et al. 1989. Biotechnol. Appl. Biochem. 11:133; Wagner E. et al. 1992. Proc. Natl. Acad. Sci. 88:4255).

The optimal protocol for uptake of oligonucleotides will depend upon a number of factors, the most crucial being the type of cells that are being used. Other factors that are important in uptake include, but are not limited to, the nature and concentration of the oligonucleotide, the confluence of the cells, the type of culture the cells are in (e.g., a suspension culture or plated) and the type of media in which the cells are grown.

A considerable number of transfection reagents are know in the art and include compositions such as LIPOFECTAMINE 2000™ and related compositions, available from Invitrogen Corporation (cat. no. 11668-019, 11668-027 and 12566-014).

### Virus-Like Particles

VLPs suitable for use in practicing the invention can be formed by any number of methods. Typically, viral components will be selected which allow for the production of VLPs having one or more of the following properties. (1) The ability to bind or act as a vehicle for one or more specified compounds. (2) The ability to enter one or more cells or cell types (e.g., endothelial, blood, neuronal, muscular, etc.) or cells in different stages of development (e.g., stem cells, progenitor cells, actively dividing cells, non-dividing cells, etc.). (3) The ability to enter cells of an organism of one or more type (e.g., Escherichia coli, primate, rodent, plant, etc.) or species (e.g., C. elegans, human, mouse, rat, etc.).

In some instance, VLPs will be formed from wild-type viral components. In other instances, one or more VLP component will be of a non-wild type form. Instances in which it may be desirable to use a non-wild-type form of a viral component include those where it is desirable to alter one or more property of the viral component to introduce, remove, enhance or diminish one or more properties. As an example, some sequence specificities for binding of the HIV-1 nucleopsid protein to oligonucleotides have been determined (see. e.g., Fisher et al., Nucl. Acids. Res. 34:472-484 (2006). In particular, zinc finger regions of the nucleocapsid protein have been shown to exhibit stable sequence-specific binding affinity for oligonucleotides containing at least 5 bases of the repeating sequence d(TG)n. Thus, in specific embodiments, VLPs of the invention may contain a wild-type HIV-1 nucleocapsid protein and a nucleic acid molecule containing a (TG)n repeat sequence. VLPs of the invention may contain a non-wild-type HIV-1 nucleocapsid protein which has been altered such that it retains binding affinity for a (TG)n repeat sequence and/or has binding affinity for another sequence. Methods for generating altered proteins and identifying which altered proteins have one or more desired characteristics are know in the art and discussed elsewhere here.

Baculoviruses are large, enveloped viruses that infect arthropods. Baculoviral genomes are double-stranded DNA molecules of approximately 130 kbp in length. Baculoviruses have gained widespread use as systems in which to express proteins, particularly proteins from eukaryotic organisms (e.g., mammals), as the insect cells used to culture the virus may more closely mimic the post-translational modifications (e.g., glycosylation, acylation, etc.) of the native organism.

Numerous expression systems utilizing recombinant baculoviruses have been developed. General methods for constructing recombinant baculoviruses for expression of heterologous proteins may be found in Piwnica-Worms, et al., (1997) Expression of Proteins in Insect Cells Using Baculovirus Vectors, in Current Protocols in Molecular Biology, Chapter 16, pp. 16.9.1 to 16.11.12, Ausubel, et al. Eds., John Wiley & Sons, Inc. Other expression systems are known, for example, U.S. Pat. No. 6,255,060, issued to Clark, et al. discloses a baculoviral expression system for expressing nucleotide sequences that include a tag. U.S. Pat. No. 5,244,805, issued to Miller, discloses a baculoviral expression system that utilizes a modified promoter not naturally found in baculoviruses. U.S. Pat. No. 5,169,784, issued to Summers, et al. discloses a baculoviral expression system that utilizes dual promoters (e.g., a baculoviral early promoter and a baculoviral late promoter). U.S. Pat. No. 5,162,222, issued to Guarino, et al. discloses a baculoviral expression system that can be used to create stable cells lines or infectious viruses expressing heterologous proteins from a baculoviral immediate-early promoter (i.e., IEN). U.S. Pat. No. 5,155,037, issued to Summers, et al. discloses a baculoviral expression system that utilizes insect cell secretion signal to improve efficiency of processing and secretion of heterologous genes. U.S. Pat. No. 5,077,214, issued to Guarino, et al. discloses the use of baculoviral early gene promoters to construct stable cell lines expression heterologous genes. U.S. Pat. No. 4,879,239, issued to Smith, et al. discloses a baculoviral expression system that utilizes the baculoviral polyhedrin promoter to control the expression of heterologous genes.

Various methods of constructing recombinant baculoviruses have been used. A frequently used method involves transfecting baculoviral DNA and a plasmid containing baculoviral sequences flanking a heterologous sequence. Homologous recombination between the plasmid and the baculoviral genome results in a recombinant baculovirus containing the heterologous sequences. This results in a mixed population of recombinant and non-recombinant viruses. Recombinant baculoviruses may be isolated from non-recombinant by plaque purification. Viruses produced in this fashion may require several rounds of plaque purification to obtain a pure strain. Methods to reduce the background of non-recombinant viruses produced by homologous recombination methods have been developed. For example, a linearized baculoviral genome containing a lethal deletion, BACULOGOLD™, is commercially available from BD Biosciences, San Jose, Calif. The lethal deletion is rescued by homologous recombination with plasmids containing baculoviral sequences from the polyhedrin locus.

Methods utilizing direct insertion of foreign sequences into a baculoviral genome are also known. For example, Peakman, et al. (Nucleic Acids Res 20(3):495-500, 1992) disclose the construction of baculoviruses having a lox site in the genome. Heterologous sequences may be moved into the genome by in vitro site-specific recombination between a plasmid having a lox site and the baculoviral genome in the presence of Cre recombinase. U.S. Pat. No. 5,348,886, issued to Lee, et al. discloses a baculoviral expression system that utilizes a bacmid (a hybrid molecule comprising a baculoviral genome and a prokaryotic origin of replication and selectable marker) containing a recombination site for Tn7 transposon. Prokaryotic cells carrying the bacmid are transformed with a plasmid having a Tn7 recombination site and with a plasmid expressing the activities necessary to catalyze recombination between the Tn7 sites. Heterologous sequences present on the plasmid are introduced into the bacmid by site-specific recombination between the Tn7 sites. The recombinant bacmid may be purified from the prokaryotic host and introduced into insect cells to initiate an infection. Recombinant viruses carrying the heterologous sequence are produced by the cells transfected with the bacmid.

The family Retroviridae contains three subfamilies: 1) oncovirinae; 2) spumavirinae; and 3) lentivirinae. Retroviruses (e.g., lentiviruses) are viruses having an RNA genome that replicate through a DNA intermediate. A retroviral particle contains two copies of the RNA genome and viral replication enzymes in a RNA-protein viral core. The core is surrounded by a viral envelop made up of virally encoded glycoproteins and host cell membrane. In the early steps of infection, retroviruses deliver the RNA-protein complex into the cytoplasm of the target cell. The RNA is reverse transcribed into double-stranded cDNA and a pre-integration complex containing the cDNA and the viral factors necessary to integrate the cDNA into the target cell genome is formed. The complex migrates to the nucleus of the target cell and the cDNA is integrated into the genome of the target cell. As a consequence of this integration, the DNA corresponding to the viral genome (and any heterologous sequences contained in the viral genome) is replicated and passed on to daughter cells. This makes it possible to permanently introduce heterologous sequences into cells.

A wide variety of retroviruses are known, for example, leukemia viruses such as a Moloney Murine Leukemia Virus (MMLV) and immunodeficiency viruses such as the Human Immunodeficiency Virus (HIV). Representative examples of retroviruses include, but are not limited to, the Gibbon Ape Leukemia virus (GALV), Avian Sarcoma-Leukosis Virus (ASLV), which includes but is not limited to Rous Sarcoma Virus (RSV), Avian Myeloblastosis Virus (AMV), Avian Erythroblastosis Virus (AEV) Helper Virus, Avian Myelocytomatosis Virus, Avian Reticuloendotheliosis Virus, Avian Sarcoma Virus, Rous Associated Virus (RAV), and Myeloblastosis Associated Virus (MAV).

Retroviruses have found widespread use as gene therapy vectors. To reduce the risk of transmission of the gene therapy vector, gene therapy vectors have been developed that have modifications that prevent the production of replication competent viruses once introduced into a target cell. For example, U.S. Pat. No. 5,741,486 issued to Pathak, et al. describes retroviral vectors comprising direct repeats flanking a sequence that is desired to be deleted (e.g., a cis-acting packing signal) upon reverse transcription in a host cell. Deletion of the packing signal prevents packaging of the recombinant viral genome into retroviral particles, thus preventing spread of retroviral vectors to non-target cells in the event of infection with replication competent viruses. U.S. Pat. Nos. 5,686,279, 5,834,256, 5,858,740, 5,994,136, 6,013,516, 6,051,427, 6,165,782, and 6,218,187 describe a retroviral packaging system for preparing high titer stocks of recombinant retroviruses. Plasmids encoding the retroviral functions required to package a recombinant retroviral genome are provided in trans. The packaged recombinant retroviral genomes may be harvested and used to infect a desired target cell.

The family Herpesviridae contains three subfamilies 1) alphaherpesvirinae, containing among others human herpesvirus 1; 2) betaherpesvirinae, containing the cytomegaloviruses; and 3) gammaherpesvirinae. Herpesviruses are enveloped DNA viruses. Herpesviruses form particles that are approximately spherical in shape and that contain one molecule of linear dsDNA and approximately 20 structural proteins. Numerous herpesviruses have been isolated from a wide variety of hosts. For example, U.S. Pat. No. 6,121,043 issued to Cochran, et al. describes recombinant herpesvirus of turkeys comprising a foreign DNA inserted into a non-essential region of the herpesvirus of turkeys genome; U.S. Pat. No. 6,410,311 issued to Cochran, et al. describes recombinant feline herpesvirus comprising a foreign DNA inserted into a region corresponding to a 3.0 kb EcoRI-SalI fragment of a feline herpesvirus genome, U.S. Pat. No. 6,379,967 issued to Meredith, et al., describes herpesvirus saimiri, (HVS; a lymphotropic virus of squirrel monkeys) as a viral vector; and U.S. Pat. No. 6,086,902 issued to Zamb, et al. describes recombinant bovine herpesvirus type 1 vaccines.

Herpesviruses have been used as vectors to deliver exogenous nucleic acid material to a host cell. In addition to the examples above, U.S. Pat. No. 4,859,587, issued to Roizman describes recombinant herpes simplex viruses, vaccines and methods, U.S. Pat. No. 5,998,208 issued to Fraefel, et al., describes a helper virus-free herpesvirus vector packaging system, U.S. Pat. No. 6,342,229 issued to O'Hare, et al., describes herpesvirus particles comprising fusion protein and their preparation and use and U.S. Pat. No. 6,319,703 issued to Speck describes recombinant virus vectors that include a double mutant herpesvirus such as an herpes simplex virus-1 (HSV-1) mutant lacking the essential glycoprotein gH gene and having a mutation impairing the function of the gene product VP16.

RNA viruses, such as those of the families Flaviviridae and Togaviridae have also been used to deliver exogenous nucleic acids to target cells. For example, members of the genus alphavirus in the family Togaviridae have been engineered for the high level expression of heterologous RNAs and polypeptides (Frolov et al., Proc. Natl. Acad. Sci. U.S.A. 93: 11371-11377 (1996)). Alphaviruses are positive stranded RNA viruses. A single genomic RNA molecule is packaged in the virion. RNA replication occurs by synthesis of a full-length minus strand RNA intermediate that is used as a template for synthesis of positive strand genomic RNA as well for synthesis of a positive strand sub-genomic RNA initiated from an internal promoter. The sub-genomic RNA can accumulate to very high levels in infected cells making alphaviruses attractive as transient expression systems. Examples of alphaviruses are Sindbis virus and Semliki Forest Virus. Kunjin virus is an example of a flavivirus. Sub-genomic replicons of Kunjin virus have been engineered to express heterologous polypeptides (Khromykh and Westaway, J. Virol. 71: 1497-1505 (1997)). The genomic RNA of both flaviviruses and togaviruses are infectious; transfection of the naked genomic RNA results in production of infective virus particles.

Adenoviruses are non-enveloped viruses with a 36 kb DNA genome that encodes more than 30 proteins. At the ends of the genome are inverted terminal repeats (ITRs) of approximately 100-150 base pairs. A sequence of approximately 300 base pairs located next to the 5′-ITR is required for packaging of the genome into the viral capsid. The genome as packaged in the virion has terminal proteins covalently attached to the ends of the linear genome.

The genes encoded by the adenoviral genome are divided into early and late genes depending upon the timing of their expression relative to the replication of the viral DNA. The early genes are expressed from four regions of the adenoviral genome termed E1-E4 and are transcribed prior to onset of DNA replication. Multiple genes are transcribed from each region. Portions of the adenoviral genome may be deleted without affecting the infectivity of the deleted virus. The genes transcribed from regions E1, E2, and E4 are essential for viral replication while those from the E3 region may be deleted without affecting replication. The genes from the essential regions can be supplied in trans to allow the propagation of a defective virus. For example, deletion of the E1 region of the adenoviral genome results in a virus that is replication defective. Viruses deleted in this region are grown on 293 cells that express the viral E1 genes from the genome of the cell.

In addition to permitting the construction of a safer, replication-defective viruses, deletion and complementation in trans of portions of the adenoviral genome and/or deletion of non-essential regions make space in the adenoviral genome for the insertion of heterologous DNA sequences. The packaging of viral DNA into a viral particle is size restricted with an upper limit of approximately 38 kb of DNA. In order to maximize the amount of heterologous DNA that may be inserted and packaged, viruses have been constructed that lack all of the viral genome except the ITRs and packaging sequence (see, U.S. Pat. No. 6,228,646). All of the viral functions necessary for replication and packaging are provided in trans from a defective helper virus that is deleted in the packaging signal.

Recombinant adenoviruses have been used as a gene transfer vectors both in vitro and in vivo. Their principal attractions as a gene transfer vector are their ability to infect a wide variety of cells including dividing and non-dividing cells and their ability to be grown in cell culture to high titers. A number of systems to insert heterologous DNA into the adenoviral genome have been developed. The adenoviral genome has been inserted into a yeast artificial chromosome (YAC, see Ketner, et al., PNAS 91:6186-90, 1994). Mutations may be introduced into the genome by transfecting a mutation-containing plasmid into a yeast cell that contains the adenoviral YAC. Homologous recombination between the YAC and the plasmid introduces the mutation into the adenoviral genome. The adenoviral genome can be removed from the YAC by restriction digest and the genome released by restriction digest is infectious when transfected into host cells. A similar system using two plasmids has been developed in E. coli (see Crouzet, et al., PNAS 94:1414-1419, 1997, and U.S. Pat. No. 6,261,807). In this system, the adenoviral genome is introduced into a inc-P derived replicon. Mutations are introduced by homologous recombination with a plasmid containing a ColE1 origin of replication. The ITRs in the inc-P plasmid are flanked by a restriction site not present in the rest of the viral genome, thus, infectious DNA can be liberated from the plasmid by restriction digest.

A number of viruses containing recombination site sequences and/or encoding recombinases have been prepared. For example, the Cre recombinase has been expressed from recombinant adenovirus and used to excise fragments from a mouse genome that were flanked with lox sites (see Wang, et al., PNAS 93:3932-3936, 1996). U.S. Pat. No. 6,156,497 describes a system for constructing adenoviral genomes utilizing a first nucleic acid having an ITR, packaging signal, DNA of interest, and recombination site and a second nucleic acid having a recombination site and an ITR to which is bound a terminal protein. In the presence of recombinase, the two molecules are joined to form an infectious viral DNA.

Adenoviridae is a family of DNA viruses first isolated in 1953 from tonsils and adenoidal tissue of children. Six sub-genera (A, B, C, D, E, and F) and more than 49 serotypes of adenoviruses have been identified as infectious agents in humans. Although a few isolates have been associated with tumors in animals, none have been associated with tumors in humans. The adenoviral vectors most often used for gene therapy belong to the subgenus C, serotypes 2 or 5 (Ad2 or Ad5). These serotypes have not been associated with tumor formation. Infection by Ad2 or Ad5 results in acute mucous-membrane infection of the upper respiratory tract, eyes, lymphoid tissue, and mild symptoms similar to those of the common cold. Exposure to C type adenoviruses is widespread in the population with the majority of adults being seropositive for this type of adenovirus.

Adenovirus virions are icosahedrons of 65 to 80 nm in diameter containing 13% DNA and 87% protein. The viral DNA is approximately 36 kb in length and is naturally found in the nucleus of infected cells as a circular episome held together by the interaction of proteins covalently linked to each of the 5′ ends of the linear genome. The ability to work with functional circular clones of the adenoviral genome greatly facilitated molecular manipulations and allowed the production of replication defective vectors.

Two aspects of adenoviral biology are typically important for the production of replication incompetent adenoviral vectors. First is the ability to have essential regulatory proteins produced in trans, and second is the inability of adenovirus cores to package more than 105% of the total genome size. The first was originally exploited by the generation of 293 cells, a transformed human embryonic kidney cell line with stably integrated adenoviral sequences from the left-hand end (0-11 map units) comprising the E1 region of the viral genome. These cells provide the E1A gene product in trans and thus permit production of virions with genomes lacking E1A. Such virions are considered replication deficient since they can not maintain active replication in cells lacking the E1A gene (although replication may occur in high MOI conditions). 293 cells are permissive for the production of these replication deficient vectors and have been utilized in all approved protocols that use adenoviral vectors.

The second was exploited by creating backbones that exceed the 105% limit to force recombination with shuttle plasmids carrying the desired transgene. Most currently used adenoviral vector systems are based on backbones of subgroup C adenovinis, serotypes 2 or 5. Deleting regions E1/E3 alone or in combination with E2/E4 produced first- or second-generation replication-defective adenoviral vectors, respectively. As mentioned above, the adenovinis virion can package up to 105% of the wild-type genome, allowing for the insertion of approximately 1.8 kb of heterologous DNA. The deletion of E1 sequences adds another 3.2 kb, while deletion of the E3 region provides an additional 3.1 kb of foreign DNA space. Therefore, E1 and E3 deleted adenoviral vectors provide a total capacity of approximately 8.1 kb of heterologous DNA sequence packaging space.

Adenoviruses have been extensively characterized and make attractive vectors for gene therapy because of their relatively benign symptoms even as wild type infections, their ease of manipulation in vitro, the ability to consistently produce high titer purified virus, their ability to transduce quiescent cells, and their broad range of target tissues. In addition, adenoviral DNA is not incorporated into host cell chromosomes minimizing concerns about insertional mutagenesis or potential germ line effects. This has made them very attractive vectors for tumor gene therapy protocols and other protocols in which transient expression may be desirable. However, these vectors are not very useful for metabolic diseases and other application for which long-term expression may be desired. Human subgroup C adenoviral vectors lacking all or part of E1A and E1B regions have been evaluated in Phase I clinical trials that target cancer, cystic fibrosis, and other diseases without major toxicities being described. Disadvantages of adenoviral-based vectors systems include a limited duration of transgene expression and the host's immune response to the expression of late viral gene products.

Kochanek and colleagues recently generated a new adenoviral vector with increased insert capacity and to specifically address the issues of immunogenicity of late viral gene expression. (See Volpers and Kochaneck, J. Gene Med. 6 Suppl. 1:S164-71 (2004).) This large capacity vector, designated the delta vector, can package up to 30 kb of foreign DNA and expresses no viral genes. The vector can be propagated in the same 293 cells with the additional viral functions provided by a first generation helper vector. A smaller genome in the delta vector compared to that of the helper vector gives them different buoyant densities and allows for purification by CsCl banding. With this method of production, the residual helper vector level is 1% or less in the purified stock. The titer of the purified delta vector achieved in the original report was 1.4×10 infectious units (i.u.)/ml with a total yield of 4.9×10 i.u. from 1.6×10 293 cells. The integrity of the vector particles was investigated by electron microscopy and found morphologically identical to helper virus particles.

After adenoviral vector mediated gene transfer, the viral-transgene genome is maintained epichromosomally in target cells. Thus, with proliferation of the transduced cells, vector sequences are lost, resulting in transgene expression of limited duration. To address the issue of transient gene expression associated with adenoviral vectors, it is advantageous to have a chimeric vector system that combines the high in vivo gene delivery efficiency of recombinant adenoviral vectors with the integrative capabilities of retroviral vectors.

Retroviral Vectors

Retroviruses comprise the most intensely scrutinized group of viruses in recent years. The Retroviridae family has traditionally been subdivided into three sub-families largely based on the pathogenic effects of infection, rather than phylogenetic relationships. The common names for the sub-families are tumor- or onco-viruses, slow- or lenti-viruses and foamy- or spuma-viruses. The latter have not been associated with any disease and are the least well known. Retroviruses are also described based on their tropism: ecotropic, for those which infect only the species of origin (or closely related species amphotropic, for those which have a wide species range normally including humans and the species of origin, and xenotrophic, for those which infect a variety of species but not the species of origin.

Tumor viruses comprise the largest of the retroviral sub-families and have been associated with rapid (e.g., Rous Sarcoma virus) or slow (e.g., mouse mammary tumor virus) tumor production. Onco-viruses are sub-classified as types A, B, C, or D based on the virion structure and process or maturation. Most retroviral vectors developed to date belong to the C type of this group. These include the Murine leukemia viruses and the Gibbon ape virus, and are relatively simple viruses with few regulatory genes. Like most other retroviruses, C type based retroviral vectors require target cell division for integration and productive transduction.

An important exception to the requirement for cell division is found in the lentivirus sub-family. The human immunodeficiency virus (HIV), the most well known of the lentiviruses and etiologic agent of acquired immunodeficiency syndrome (AIDS), was shown to integrate in non-dividing cells. Although the limitation of retroviral integration to dividing cells may be a safety factor for some protocols such as cancer treatment protocols, it is probably the single most limiting factor in their utility for the treatment of inborn errors of metabolism and degenerative traits.

Examples of retroviruses are found in almost all vertebrates, and despite the great variety of retroviral strains isolated and the diversity of diseases with which they have been associated, all retroviruses share similar structures, genome organizations, and modes of replication. Retroviruses are enveloped RNA viruses approximately 100 nm in diameter. The genome consists of two positive RNA strands with a maximum size of around 10 kb. The genome is organized with two long terminal repeats (LTR) flanking the structural genes gag, pol, and env. The presence of additional genes (regulatory genes or oncogenes) varies widely from one viral strain to another. The env gene codes for proteins found in the outer envelope of the virus. These proteins convey the tropism (species and cell specificity) of the virion. The pol gene codes for several enzymatic proteins important for the viral replication cycle. These include the reverse transcriptase, which is responsible for converting the single stranded RNA genome into double stranded DNA, the integrase which is necessary for integration of the double stranded viral DNA into the host genome and the proteinase which is necessary for the processing of viral structural proteins. The gag, or group specific antigen gene, encodes the proteins necessary for the formation of the virion nucleocapsid.

Recombinant retroviruses are considered to be the most efficient vectors for the stable transfer of genetic material into actively replicating mammalian cells. The retroviral vector is a molecularly engineered, non-replicating delivery system with the capacity to encode approximately 8 kb of genetic information. To assemble and propagate a recombinant retroviral vector, the missing viral gag-pol-env functions must be supplied in trans.

Since their development in the early 1980's, vectors derived from type C retroviruses represent some of the most useful gene transfer tools for gene expression in human and mammalian cells. Their mechanisms of infection and gene expression are well understood. The advantages of retroviral vectors include their relative lack of intrinsic cytotoxicity and their ability to integrate into the genome of actively replicating cells. However, there are a number of limitations for retroviruses as a gene delivery system including a limited host range, instability of the virion, a requirement for cell replication, and relatively low titers.

Although amphotropic retroviruses have a broad host range, some cell types are relatively refractory to infection. One strategy for expanding the host range of retroviral vectors has been to use the envelope proteins of other viruses to encapsidate the genome and core components of the vector. Such pseudotyped virions exhibit the host range and other properties of the virus from which the envelope protein was derived. The envelope gene product of a retrovirus can be replaced by VSV-G to produce a pseudotyped vector able to infect cells refractory to the parental vector. While retroviral infection usually requires specific interaction between the viral envelope protein and specific cell surface receptors, VSV-G interacts with a phosphatidyl serine and possibly other phospholipid components of the cell membrane to mediate viral entry by membrane fusion. Since viral entry is not dependent on the presence of specific protein receptors, VSV has an extremely broad host-cell range. In addition, VSV can be concentrated by ultracentrifugation to titers greater than 10⁹ colony forming units (cfa)/ml with minimal loss of infectivity, while attempts to concentrate amphotropic retroviral vectors by ultracentrifugation or other physical means has resulted in significant loss of infectivity with only minimal increases in final titer.

However, since VSV-G protein mediates cell fusion it is toxic to cells in which it is expressed. This has led to technical difficulties for the production of stable pseudotyped retroviral packaging cell lines. One approach for production of VSV-G pseudotyped vector particles has been by transient expression of the VSV-G gene after DNA transfection of cells that express a retroviral genome and the gaglpol components of a retrovirus. Generation of vector particles by this method is cumbersome, labor intensive, and not easily scaled up for extensive experimentation. Recently, Yoshida et al. produced VSV-G pseudotyped retroviral packaging through adenovirus-mediated inducible gene expression. Tetracycline (tet)-controllable expression was used to generate recombinant adenoviruses encoding the cytotoxic VSV-G protein. A stably transfected retroviral genome was rescued by simultaneous transduction with three recombinant adenoviruses: one encoding the VSV-G gene under control of the tet promoter, another the retroviral gag/pol genes, and a third encoding the tetracycline transactivator gene. This resulted in the production of VSV-G pseudotyped retroviral vectors. Although both of these systems produce pseudotyped retroviruses, both are unlikely to be amenable to clinical applications that demand reproducible, certified vector preparation.

Another limitation for the use of retroviral vectors for human gene therapy applications has been their short in vivo half-life. This is partly due to the fact that human and non-human primate sera rapidly inactivate type C retroviruses. Viral inactivation occurs through an antibody-independent mechanism involving the activation of the classical complement pathway. The human complement protein Clq was shown to bind directly to MLV virions by interacting with the transmembrane envelope protein p15E. An alternative mechanism of complement inactivation has been suggested based upon the observation that surface glycoproteins generated in murine cells contain galactose-.alpha.-(1,3)-galactose sugar moieties. Humans and other primates have circulating antibodies to this carbohydrate moiety. Rother and colleagues propose that these anti-carbohydrate antibodies are able to fix complement, which leads to subsequent inactivation of murine retroviruses and murine retrovirus producer cells by human serum. Therefore, inactivation of retroviral vectors by complement in human serum is determined by the cell line used to produce the vectors and by the viral envelope components. It has been demonstrated that the human 293 and HOS cell lines are capable of generating amphotropic retroviral vectors that are relatively resistant to inactivation by human serum. In similar experiments, it has been found that VSV-G pseudotyped retroviral vectors produced in a 293 packaging cell line were significantly more resistant to inactivation by human serum than commonly used amphotropic retroviral vectors generated in .PSI.CRIPLZ cells (a NIH-3T3 murine-based producer cell line). The cell lines used to produce the retroviral vectors by the systems described herein could easily select for their resistance to complement. In addition, in vivo produced vectors would overcome the issue of complement inactivation.

Bilbao and colleagues also used a multiple adenoviral vector system to transiently transduce cells to produce retroviral progeny. (See Bilbao et al., FASEB J. 11(8):624-34 (1997).) An adenoviral vector encoding a retroviral backbone (the LTRs, packaging sequence, and a reporter gene) and another adenoviral vector encoding all of the trans acting retroviral functions (the CMV promoter regulating gag, pol, and env) accomplished in vivo gene transfer to target parenchymal cells at high efficiency rendering them transient retroviral producer cells. Athymic mice xenografted orthotopically with the human ovary carcinoma cell line SKOV3 and then challenged intraperitoneally with the two adenoviral vector systems demonstrated the concept that adenoviral transduction had occurred with the in situ generation of retroviral particles that stably transduced neighboring cells in the target parenchyma. These systems established the foundation that adenoviral vectors may be utilized to render target cells transient retroviral vector producer cells, however, they are unlikely to be easily amenable to clinical applications that demand reproducible, certified vector preparation because of the stochastic nature for multiple vector transduction of single cells in vivo. Thus, the invention includes methods for producing VLPs which combine components from different viruses, including viruses of different classes (e.g., DNA and RNA viruses).

In PCT Patent No. WO 97/25446, methods and vectors are described directed toward generating adenoviral vectors at high titers in the absence of the requirement for selectable markers and screening procedures. In a specific embodiment a hybrid adenoviral/retroviral vector is generated which creates producer cells from transduced cells for the purpose of permanent integration of a gene of interest. In this method, a first polynucleotide containing a 5′ adenoviral inverted terminal repeat, retroviral LIR sequences flanking a heterologous sequence of interest, gag/pol and env sequences outside of the retroviral LTR sequences, and a recombinase sequence are transfected with a second polynucleotide containing a 3′ adenoviral inverted terminal repeat and a recombinase site. A recombinase is provided on a third polynucleotide or is contained in a cell. Upon transfection with the multiple polynucleotides and action by the recombinase, a complete adenoviral sequence is produced containing retroviral sequences including the LTRs, gag/pol and env.

In PCT Patent No. WO 98/22143, a system for in vivo gene delivery employing a chimeric vector wherein in situ production of retroviral particles inside a cell by the generation of replication-defective adenoviral vectors which contain either the retroviral genes gag, pol and env or the retroviral LTR sequences flanking a gene of interest. The presence of these elements on multiple vectors requires the manipulation of multiple species for transfection of cells and subsequent generation of producer cells.

In PCT Patent No. WO 99/55894, vectors and methods are described therein directed to a combination of adenoviral and retroviral vectors for the generation of packaging cells for delivery of a therapeutic gene. A retroviral vector delivers a gene of interest, and an adenovirus-based delivery system delivers gag, pol and env. Again, multiple vectors are employed for transfer of a sequence of interest and subsequent production of retroviral producer cells.

Kits and Instructions:

The invention also provides kits. Kits of the invention may be designed to allow users to produce or use VLPs which contain one or more compounds. Kits of the invention may also contain one or more VLPs which contain one or more compound.

In various aspects, a kit of the invention may contain one or more (e.g., one, two, three, four, five, six, seven, etc.) of the following components: (1) one or more sets of instructions, including, for example, instructions for performing methods of the invention or for preparing and/or using compositions of the invention; (2) one or more cells, including, for example, one or more mammalian cells, for example, cells that are adapted for growth in a tissue culture medium, (3) one or more oligonucleotide or double stranded nucleic acid molecule (including one or more control nucleic acid molecule, as described elsewhere herein); (4) one or more container containing water (e.g., distilled water) or other aqueous or liquid material; (5) one or more containers containing one or more buffers, which can be buffers in dry, powder form or reconstituted in a liquid such as water, including in a concentrated form such as 2×, 3×, 4×, 5×, etc.); and/or (6) one or more containers containing one or more salts (e.g., sodium chloride, potassium chloride, magnesium chloride, which can be in a dry, powder form or reconstituted in a liquid such as water).

A kit of the invention can include an instruction set, or the instructions can be provided independently of a kit. Such instructions may provide information regarding how to make or use one or more of the following items: (1) one or more control nucleic acid molecule (e.g., a nucleic acid molecule which may be used as a transfection control); (2) one or more double stranded nucleic acid molecule, as described elsewhere herein (e.g., a double stranded nucleic acid molecule which is capable of “knocking-down” expression of a gene where introduced into a eukaryotic cell); (3) one or more cell lines that contain a gene the expression of which is to be knocked down (e.g., pre-transfection growth conditions; transfection protocols; post-transfection growth conditions); (4) one or more dyes for distinguishing live from dead cells (e.g., Red Dead stain (see Invitrogen Corp., cat. no. L23102), Trypan Blue, etc.), and/or (5) one or more sets of instructions for using kit components.

Instructions can be provided in a kit, for example, written on paper or in a computer readable form provided with the kit, or can be made accessible to a user via the internet, for example, on the world wide web at a URL (uniform resources link; i.e., “address”) specified by the provider of the kit or an agent of the provider. Such instructions direct a user of the kit or other party of particular tasks to be performed or of particular ways for performing a task. In one aspect, the instructions instruct a user of how to perform methods of the invention. In a specific aspect, the instructions can, for example, instruct a user of a kit as to reaction conditions for knocking-down gene expression, including, for example, buffers, temperature, and/or time periods of incubations for using nucleic acid molecules described herein. Instructions of the invention can be in a tangible form, for example, printed or otherwise imprinted on paper, or in an intangible form, for example, present on an internet web page at a defined and accessible URL. Thus, the invention includes instructions for performing methods of the invention and/or for preparing compositions of the invention. While the instructions themselves are one aspect of the invention, the invention also includes the instructions in tangible form. Thus, the invention includes computer media (e.g., hard disks, floppy disks, CDs, etc.) and sheets of paper (e.g., a single sheet of paper, a booklet, etc.) which contain the instructions.

It will be recognized that a full text of instructions for performing a method of the invention or, where the instructions are included with a kit, for using the kit, need not be provided. One example of a situation in which a kit of the invention, for example, would not contain such full length instructions is where the provided directions inform a user of the kits where to obtain instructions for practicing methods for which the kit can be used. Thus, instructions for performing methods of the invention can be obtained from internet web pages, separately sold or distributed manuals or other product literature, etc. The invention thus includes kits that direct a kit user to one or more locations where instructions not directly packaged and/or distributed with the kits can be found. Such instructions can be in any form including, but not limited to, electronic or printed forms.

The invention is further illustrated by the following examples, which should not be construed as further limiting.

### EXAMPLES

### Example 1

### Gene Silencing of Transiently Expressed lacZ Using Lentiviral Delivery of shRNAs

Generation of lentiviral particles containing lacZ shRNAs. To demonstrate that shRNAs can be packaged into lentiviral particles and are delivered to target cells, lentivirus are generated in cells expressing shRNAs directed towards the lacZ message. The shRNA expression vector, pENTR/U6 (Invitrogen Corp., cat. no. K4944-00), is used to express the lacZ shRNA in transfected cells. The target sequence of the shRNA that corresponds to lacZ message is 5′-CGACTACACAAATCAGCGATTTC-3′ (SEQ ID NO: 1). The resulting shRNA has a four base pair loop.

Virus particles are produced by transfecting expression vectors encoding the HIV-1 gag-pol (pLP1) or HIV-1 gag-pro (pGag-Pr) into 293-FT cells (Invitrogen Corp., cat. no. R700-07). pGag-Pr includes the HIV gag-pol gene under control of a CMV promoter. An amber stop codon was inserted immediately downstream of the last codon in the protease gene. Expression from pGag-Pr produces Gag and Gag-Pr proteins. Gag-Pr is produced by a ribosomal frameshift within the modified gag-pol gene. Both of these vectors include the rev responsive element (RRE) and therefore require co-transfection of the Rev expression vector (pLP2) for efficient expression. Cells are also co-transfected with pLP/VSV-G which encodes the vesicular stomatitis virus glycoprotein (VSV-G).

Briefly, 293-FT cells are plated at 90% in a T175 cm² flask 1 day before transfection. All plasmids (18 μg of each), pENTR/U6/lacZ, pLP1 or pGag-Pr, pLP2, and pLP/VSV-G are co-transfected into 293-FT cells using Lipofectamine 2000. Supernatants containing virus-like particles are collected 2 days post-transfection, clarified by centrifugation at 400×g for 10 min, followed by filtration through a 0.45-μm-pore-size filter (Corning Inc, NY), concentrated by ultracentrifugation for 2 hours at 27,000 rpm, resuspended in PBS, and stored at −80° C. for further experiments.

Transduction of cell lines expressed LacZ gene with shRNA-containing virus-like particles. HT1080 (ATCC No. CCL-121) or GripTite293 (Invitrogen Corp., cat. no. R795-07) cells (50% confluent) were transfected with the lacZ expression vector, pcDNA6.2/GW/V5-lacZ and luciferase expression vector pcDNA/FRT-luc. One day post transfection, these cells are plated at 1×10⁵ cells per well in 24-well plates. Following attachment to plates, the medium is replaced with 150 μl of fresh complete medium. Virus like particles (100 μl) produced in the presence or absence of the shRNA expression vector, pENTR/U6/lacZ, are added to the wells in the presence of 1 μg/ml of final concentration of polybrene. Twenty four hours later, the cells awere analyzed for β-Galactosidase System (Galacto-Light Plus, Applied Biosystems, Bedford, Mass.) and luciferase (Luciferase Reporter 1000 Assay System, Promega) activity according to manufacturer's instructions. Total protein concentration is measured by using Bio-Rad Protein Assay Dye Reagent (Bio-Rad, cat. no. 500-0006).

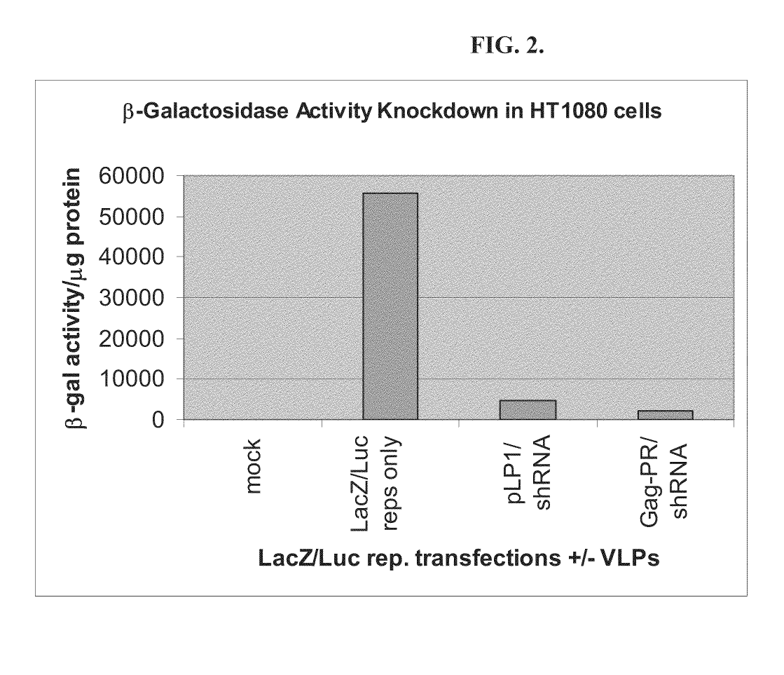

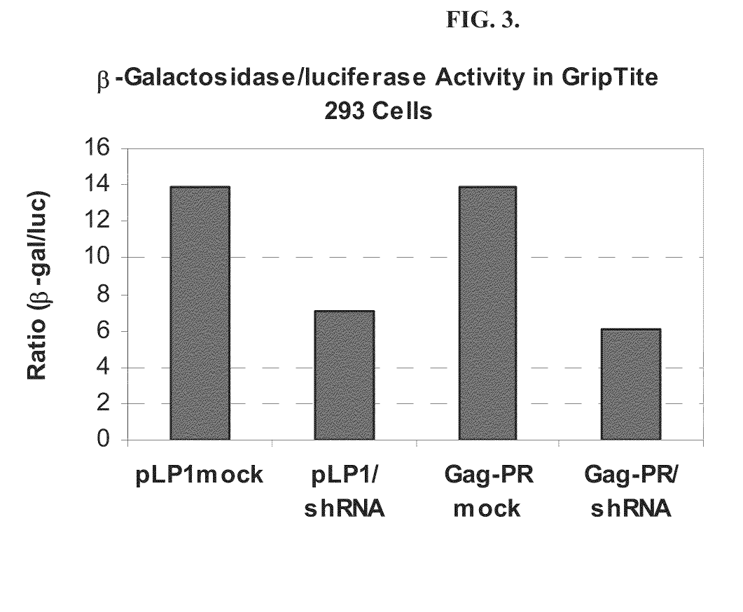

Results of the above are shown in FIGS. 2-3.

### Example 2

### Gene Silencing of lacZ Expressed in a Stable Cell Line

Generation of lentiviral particles containing lacZ shRNAs. To demonstrate that shRNAs can be packaged into lentiviral particles and are delivered to target cells, lentivirus was generated in cells expressing shRNAs directed towards the lacZ message. The shRNA expression vector, pENTR/U6 (Invitrogen), is used to express the lacZ shRNA in transfected cells. The target sequence of the shRNA that corresponds to lacZ message is 5′-CGACTACACAAATCAGCGATTTC-3′ (SEQ ID NO: 1). The resulting shRNA has a four base pair loop.

Virus particles are produced by transfecting expression vectors encoding the HIV-1 gag-pol (pLP1) or HIV-1 gag-pro (pGag-Pr) into 293-FT cells. Both of these vectors include the rev responsive element (RRE) and therefore require co-transfection of the Rev expression vector (pLP2) for efficient expression. Cells were also co-transfected with pLP/VSV-G which encodes the vesicular stomatitis virus glycoprotein (VSV-G). Briefly, 293-FT cells are plated at 90% in a T175 cm² flask 1 day before transfection. All plasmids (18 μg of each), pENTR/U6/lacZ (6 μg), pLP1 (18 μg) or pGag-Pr (18 μg), pLP2 (18 μg), and pLP/VSV-G (18 μg) are co-transfected into 293-FT cells using Lipofectamine2000. Supernatants containing virus-like particles are collected 2 days post-transfection are filtered through a 0.45-μm-pore-size filter (Corning Inc, NY), concentrated by ultracentrifugation for 2 hours at 27,000 rpm, resuspended in PBS, and stored at −80° C. for further experiments.

Western Blot Analysis of virus particles. To determine the relative amounts of virus particles produced, 10 μl of the virus prep is mixed with sample buffer, boiled for 5 minutes and separated by electrophoresis on a 10% NuPAGE® Novex Bis-Tris Gel. Gels are run at 200V for 50 minutes using MOPS buffer, then transferred to PVDF membrane and detected by a WesternBreeze® Chemiluminescent Immunodetection kit using HIV p24 antibody (Abcam).

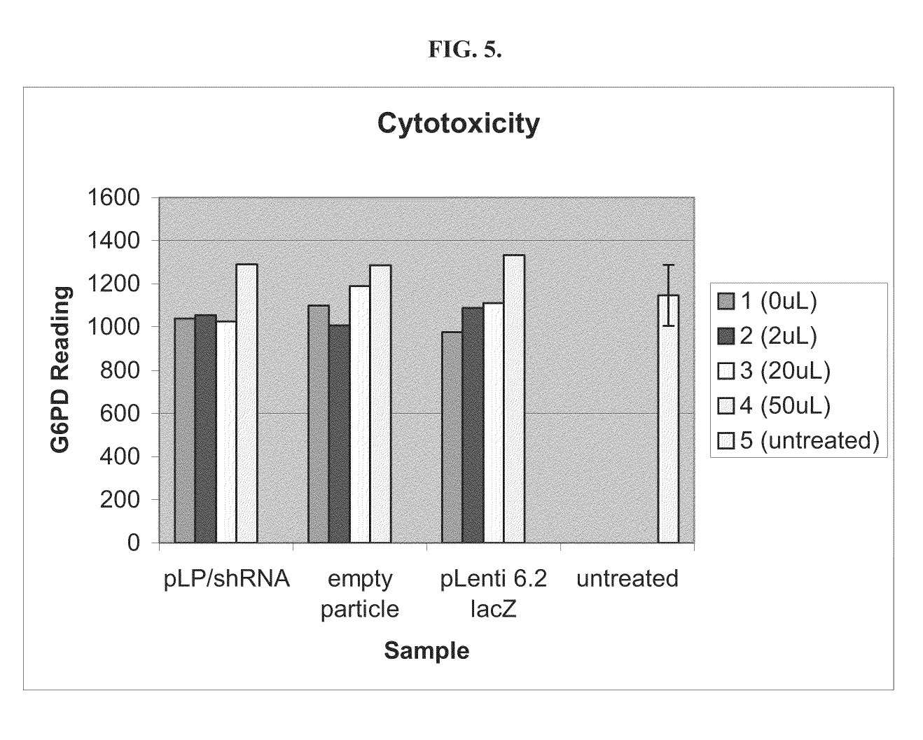

Transduction of cell lines stably expressing lacZ with shRNA-containing virus-like particles. Flp-In293 cells (3.0×10̂5 cells/well) containing an integrated copy of lacZ are transduced with virus like particles (100 μl) produced in the presence or absence of the shRNA expression vector, pENTR/U6/lacZ, in the presence of 1 μg/ml final concentration of polybrene. Virus (made with pLenti6.2/lacZ) that expresses the lacZ shRNA (as oppose to shRNA delivery) upon reverse transcription and integration is included as a positive control for message knockdown. Cytotoxicity assays (Vybrant Cytoxicity Assay Kit-G6PD release assay, Invitrogen, Carlsbad, Calif.) are performed to determine if the applied virus preps had any deleterious effects on cell growth. (FIG. 5).

The ability of the retroviral particles to knock down the lacZ message is measured by a qPCR assays 24 and 48 hours post virus addition. Cells are lysed in Lysis Solution with DDT by incubating at −80° C. for an hour then thawed to break open cell walls. RNA was isolated using an mRNA Catcher™ Plus plate (Invitrogen, Carlsbad, Calif.) according to manufacturer's recommendations. From the isolated mRNA, cDNA is produced by performing a RT-PCR reaction on the isolated mRNA using SUPERSCRIPT™ III Reverse Transcriptase (Invitrogen, Carlsbad, Calif.). The qPCR assays are performed with appropriate LacZ forward and reverse primers at (100 μM concentration) using SYBR® GreenER™ detection. GapDH and Cyclophilin are used as normalization in the qPCR assay. Each sample is run in triplicate and averaged for data point. qPCR 384-well plates are run on Applied Biosystems 7900HT Sequence Detection System qPCR machines and raw data is analyzed with SDS 2.1 software.

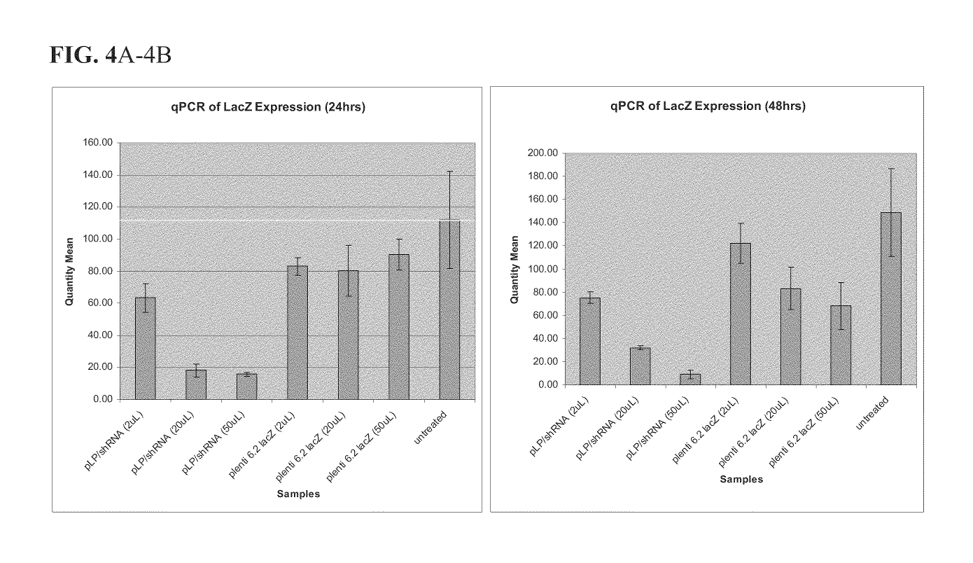

Results are shown in FIGS. 4-5.

### EQUIVALENTS

Those skilled in the art will recognize, or be able to ascertain using no more than routine experimentation, many equivalents to the specific embodiments of the invention described herein. Such equivalents are intended to be encompassed by the following claims. The entire contents of all patents, published patent applications and other references cited herein are hereby expressly incorporated herein in their entireties by reference.

1. A method of introducing an RNA molecule into a cell, the method comprising:
(a) selecting a nucleic acid of interest which is heterologous to the cell;
(b) transcribing the nucleic acid of interest to generate the RNA molecule;
(c) forming virus-like particles under conditions which result in the RNA molecule being incorporated into the virus-like particles; and
(d) contacting the cell with the virus-like particles formed in step (c),
wherein the RNA molecule does not contain a packaging signal and is less than 150 nucleotides in length.

2. The method of claim 1, wherein the RNA molecule is between 15 and 100 nucleotides in length.

3. The method of claim 1, wherein the RNA molecule is between 15 and 30 nucleotides in length.

4. The method of claim 1, wherein the virus-like particles are retroviral virus-like particles.

5. The method of claim 4, wherein the retroviral virus-like particles are generated using components from retrovirus selected from the group consisting of Moloney Murine leukemia virus and a lentivirus.

6. The method of claim 1, wherein the RNA molecule is double-stranded.

7. The method of claim 6, wherein the RNA molecule is composed of two separate RNA strands.

8. The method of claim 6, wherein the RNA molecule is composed of one RNA strand which forms a hairpin.

9. A method of inhibiting expression of a gene of interest, the method comprising:
(a) selecting the gene of interest;
(b) generating an RNA molecule with sequence complementarity to a transcript corresponding to the gene of interest;
(c) forming virus-like particles under conditions which result in the RNA molecule being incorporated into the virus-like particles; and
(d) contacting the cell with the virus-like particles formed in step (c),
wherein the RNA molecule does not contain a packaging signal and is less than 150 nucleotides in length.

10. The method of claim 9, wherein the gene of interest encodes a polypeptide.

11. The method of claim 9, wherein the RNA molecule is single stranded.

12. The method of claim 9, wherein the RNA molecule is double stranded.

13. The method of claim 9, wherein the RNA molecule is a nucleic acid molecule selected from the group consisting of:
(a) a microRNA;
(b) a short hairpin RNA; and
(c) a short interfering RNA.

14. A method for preparing virus-like particles which contains an RNA molecule, the method comprising:
(a) selecting a gene of interest;
(b) generating the RNA molecule, wherein the RNA molecule has sequence complementarity to a transcript corresponding to the gene of interest; and
(c) forming virus-like particles under conditions which result in the RNA molecule being incorporated into the virus-like particles,
wherein the RNA molecule does not contain a packaging signal and is less than 150 nucleotides in length.

15. (canceled)

16. (canceled)

17. A method for producing a virus-like particle which contains an RNA molecule, the method comprising:
(a) selecting a nucleic acid of interest;
(b) synthesizing an RNA molecule with sequence identity to the nucleic acid of interest; and
(c) forming virus-like particles under conditions which result in the RNA molecule being incorporated into the virus-like particles
wherein the RNA molecule does not contain a packaging signal and is less than 150 nucleotides in length.

18-38. (canceled)


SEQUENCE LISTING (12 sequences)

SEQ ID NO: 1 - DNA, 23 bp, Artificial Sequence
  source: /note=“Description of Artificial Sequence Synthetic oligonucleotide”
cgactacacaaatcagcgatttc

SEQ ID NO: 2 - RNA, 21 nt, Artificial Sequence
  source: /note=“Description of Artificial Sequence Synthetic oligonucleotide”
cccuucugucuugaacaugag

SEQ ID NO: 3 - DNA, 21 bp, Artificial Sequence
  source: /note=“Description of Artificial Sequence Synthetic oligonucleotide”
ctgatgttcaagacagaacgg

SEQ ID NO: 4 - DNA, 21 bp, Artificial Sequence
  source: /note=“Description of Artificial Sequence Synthetic oligonucleotide”
ctgatgttcaagacagaacgg

SEQ ID NO: 5 - DNA, 21 bp, Artificial Sequence
  source: /note=“Description of Combined DNA/RNA Molecule Synthetic oligonucleotide”
ctcauguucaagacagaaggg

SEQ ID NO: 6 - DNA, 21 bp, Artificial Sequence
  source: /note=“Description of Combined DNA/RNA Molecule Synthetic oligonucleotide”
ctcauguucaagacagaaggg

SEQ ID NO: 7 - DNA, 21 bp, Artificial Sequence
  source: /note=“Description of Combined DNA/RNA Molecule Synthetic oligonucleotide”
ctcauguucaagacagaaggg

SEQ ID NO: 8 - DNA, 21 bp, Artificial Sequence
  source: /note=“Description of Artificial Sequence Synthetic oligonucleotide”
gagtacaagttctgtcttccc

SEQ ID NO: 9 - DNA, 21 bp, Artificial Sequence
  source: /note=“Description of Artificial Sequence Synthetic oligonucleotide”
ggcaagacagaacttgtagtc

SEQ ID NO: 10 - DNA, 21 bp, Artificial Sequence
  source: /note=“Description of Artificial Sequence Synthetic oligonucleotide”
gggaagacagaacttgtactc

SEQ ID NO: 11 - DNA, 21 bp, Artificial Sequence
  source: /note=“Description of Artificial Sequence Synthetic oligonucleotide”
gggaagacagaacttgtactc

SEQ ID NO: 12 - DNA, 21 bp, Artificial Sequence
  source: /note=“Description of Artificial Sequence Synthetic oligonucleotide”
gggaagacagaacttgtactc